(19) United States
(12) Patent Application Publication
(10) Pub. No.: US 2025/0114496 A1
(43) Pub. Date: Apr. 10, 2025

(54) INSTANT ACTION AEROSOL FOR AIR SANITIZATION AND DISINFECTION

(71) Applicant: Reckitt Benckiser LLC, Parsippany, NJ (US)

(72) Inventors: Ana Maria BAGO RODRIGUEZ, Hull (GB); Martin Richard BUTLER, Hong Kong (CN); Mohammad Khalid IJAZ, Montvale, NJ (US); Anthony KEVEK, Montvale, NJ (US); Samuel James ROPER, Hull (GB); Marina Mixtro SERRASQUEIRO, Hull (GB); Mark MILLER, Montvale, NJ (US)

(21) Appl. No.: 18/982,132

(22) Filed: Dec. 16, 2024

Related U.S. Application Data
(63) Continuation-in-part of application No. PCT/US2022/033955, filed on Jun. 17, 2022.
(60) Provisional application No. 63/612,571, filed on Dec. 20, 2023.

(30) Foreign Application Priority Data
Mar. 11, 2024 (GB) 2403478.7

(51) Int. Cl. A61L 9/14; A61L 9/01; A61L 9/12

(52) U.S. Cl. CPC A61L 9/145; A61L 9/01; A61L 9/12; A61L 2209/133; A61L 2209/134; A61L 2209/135; A61L 2209/21

(57) ABSTRACT

Air decontamination products are disclosed comprising an air decontamination formulation in an aerosol canister comprising a 2-piece mechanical breakup (MB) nozzle with swirl chamber and approximately 60 psig or more of a pressurizing gas. The air decontamination formulation comprises approximately 10% to approximately 43% by weight of the formulation of dipropylene glycol. The MB nozzle comprises two stem orifices, each having a diameter ranging from approximately 0.036 inches [0.91 mm] to approximately 0.044 inches [1.12 mm]. The disclosed air decontamination formulations may sanitize or disinfect a volume of air less than or equal to 25 m³ in 0.1 to 5 minutes by providing equal to or greater than a 3 log₁₀ reduction of aerosolized bacteria, including Staphylococcus aureus and Klebsiella pneumoniae, and aerosolized enveloped viruses, including Phi6. A 3 log₁₀ reduction of aerosolized non-enveloped viruses, like MS2, occurs in 10-15 minutes.

### PRIORITY

This application is a continuation-in-part of PCT application no. PCT/US2022/33955 filed 17 Jun. 2022 and also claims priority to U.S. provisional application No. 63/612,571, filed 20 Dec. 2023, and GB application no. 2403478.7, filed 11 Mar. 2024; the contents of each are incorporated herein by reference in their entireties.

### TECHNICAL FIELD

Air decontamination products are disclosed comprising an air decontamination formulation in an aerosol canister comprising a 2-piece mechanical breakup (MB) nozzle with swirl chamber and approximately 60 psig or more of a pressurizing gas. The air decontamination formulation comprises approximately 10% to approximately 43% by weight of the formulation of dipropylene glycol. The MB nozzle comprises two stem orifices, each having a diameter ranging from approximately 0.036 inches [0.91 mm] to approximately 0.044 inches [1.12 mm]. The disclosed air decontamination formulations may sanitize or disinfect a volume of air less than or equal to 25 m³ in 0.1 to 5 minutes by providing equal to or greater than a 3 log₁₀ reduction of aerosolized bacteria, including Staphylococcus aureus and Klebsiella pneumoniae, and aerosolized enveloped viruses, including Phi6. A 3 log₁₀ reduction of aerosolized non-enveloped viruses, like MS2, occurs in 10-15 minutes.

### BACKGROUND

Indoor air can be an important vehicle for spread of a variety of human pathogens, as evidenced by the most recent COVID-19 pandemic associated with severe acute respiratory coronavirus-2 (SARS-COV-2).

U.S. Pat. No. 2,333,124 to Robertson et al. discloses methods of sterilizing air by means of glycol vapors.

U.S. Pat. No. 2,719,129 to Richardson et al. discloses liquid room and air deodorant compositions comprising 0.5-5% of a quaternary morpholinium alkyl sulfate having an alkyl radical containing 8 to 24 carbon atoms as a deodorant, about 80-95% of a liquefied normally gaseous low molecular weight halogenated hydrocarbon propellent, and a sufficient amount of a partial ester of a polyhydric alcohol and a higher fatty acid having about 8 to 24 carbon atoms.

U.S. Pat. No. 8,465,728 to S.C. Johnson & Son, Inc. discloses an air treating composition for eliminating airborne malodors and/or sanitizing air in combination with a spray valve and actuator and spray performance parameters providing maximum dispersion of the composition. Col. 3, lines 21-23 states that no specific standards or methods for evaluating air sanitizers have been adopted by the US government.

Various analytical methods and air samplers have been used to characterize airborne pathogens and overcome the challenges of collecting and analyzing them. See, e.g., Sattar et al., Spread of viral infections by aerosols, Crit Rev Environ Control, 1987, 17:89-131; Yates et al., Manual of environmental microbiology, 4^{th} Edition, Washington (DC): ASM Press: 2015 p. 3.2.7-1-24; Verreault et al., Design of an environmentally controlled rotating chamber for bioaerosol aging studies, Inhal Toxicol 2014; 26:554-8; ljaz et al, Survival characteristics of airborne human coronavirus 229E, J Gen Virol., 1985 December, 66 (Pt 12), 2743-8; and Ijaz et al., Development of methods to study the survival of airborne viruses, J Virol Methods, 1987 Nov. 18 (2-3), 87-106.

In DIS/TSS-11 from 3 Sep. 1980, the US Environmental Protection Agency (EPA) advised that quantitative chemical determinations using an air sampling device must be performed on products containing at least 5% glycols (triethylene, dipropylene, and/or propylene glycols) to show that 50% glycol vapor saturation or more is achieved in the enclosed experimental room or chamber.

The EPA's Office of Chemical Safety and Pollution Prevention (OCSPP) issued guidance to address efficacy testing for antimicrobial pesticides intended to be used for treatment of air to temporarily reduce the number of airborne bacteria on 21 Dec. 2012 as OCSU.S. Plant Pat. No. 810,2500. The guidance states that successful test results demonstrate a viable bacteria count reduction of ≥99.9 percent (a 3 log₁₀ reduction) over a parallel untreated control, after correcting for settling rates, in the air of the test enclosure with each of the required test bacteria.

Both EPA guidelines also note that there is no standard test method for evaluating effectiveness of antimicrobial pesticides to temporarily reduce airborne bacteria.

In GB Pat 2,608,465 to Reckitt Benckiser LLC, Applicant discovered formulations having 28% wt to 38% wt dipropylene glycol were effective in microbicidal activity against airborne pathogens. In the Examples, Formulation D had 15% by weight dipropylene glycol, but failed to provide significant microbicidal activity. Applicant initially assumed that the concentration was too low to provide sufficient vapor pressure of the dipropylene glycol.

A need remains to develop microbicidal formulations demonstrating efficacy against airborne pathogens.

### BRIEF SUMMARY

Air decontamination products are disclosed comprising an aerosol canister comprising (a) a 2-piece mechanical break-up (MB) nozzle with swirl chamber, (b) an air decontamination formulation, and (c) a propellant. The air decontamination formulation comprises or consists essentially of approximately 10% to approximately 43% by weight of the formulation of dipropylene glycol, alcohol, and water. The propellant preferably is a liquified petroleum gas comprising a blend of propane and butane sufficient to produce at least 60 psig of pressure. The MB nozzle comprises two stem orifices having a diameter ranging from approximately 0.036 inches [0.91 mm] to approximately 0.044 inches [1.12 mm].

Alternatively, air decontamination products are disclosed comprising an aerosol canister comprising (a) a 2-piece mechanical break-up (MB) nozzle with swirl chamber, (b) an air decontamination formulation, and (c) a propellant. The air decontamination formulation comprises or consists essentially of approximately 10% to approximately 43% by weight of the formulation of dipropylene glycol; approximately 0.05% to approximately 0.2% by weight of the formulation of an odor neutralizing cationic surfactant; approximately 28% to approximately 50% by weight of the formulation of an alcohol; and approximately 14% to approximately 33% by weight of the formulation of water. The propellant preferably is a liquified petroleum gas comprising a blend of propane and butane sufficient to produce at least 60 psig of pressure. The MB nozzle comprises two stem orifices having a diameter ranging from approximately 0.036 inches [0.91 mm] to approximately 0.044 inches [1.12 mm].

Alternatively, the air decontamination formulations contained in the disclosed air decontamination products consist of approximately 10% to approximately 43% by weight of the formulation of dipropylene glycol; approximately 0.05% to approximately 0.2% by weight of the formulation of an odor neutralizing cationic surfactant; approximately 28% to approximately 50% by weight of the formulation of an alcohol, preferably ethanol; approximately 14% to approximately 33% by weight of the formulation of water; approximately 0.043% to approximately 0.43% by weight of the formulation of a corrosion inhibitor; approximately 0.043% to approximately 0.43% by weight of the formulation of a pH adjuster; optionally 0% to approximately 0.28% by weight of the formulation of a defoaming agent; and optionally 0% to approximately 0.43% by weight of the formulation of dye, fragrance, or both.

The disclosed air decontamination products may comprise one or more of the following aspects:
approximately 70% by weight to approximately 80% by weight of (b) the air decontamination formulation, based on the combined weight of the air decontamination formulation and the propellant;
approximately 20% by weight to approximately 30% by weight of (c) the propellant, based on the combined weight of the air decontamination formulation and the propellant;
approximately 20% by weight to approximately 30% by weight of (c) the liquid petroleum gas, based on the combined weight of the air decontamination formulation and the liquid petroleum gas;
the air decontamination formulation being a single phase liquid;
the formulation containing a ratio of alcohol:water of approximately 1:1.3;
the formulation containing a ratio of dipropylene glycol:water:alcohol of approximately 1:2:2.3;
the propellant being a blend of propane and isobutane;
the product being substantially free of dimethyl ether propellant;
the odor neutralizing cationic surfactant being a quaternary morpholinium alkyl sulfate compound;
the quaternary morpholinium alkyl sulfate compound being selected from the group consisting of soyethyl morpholinium ethosulfate, cetethyl morpholinium ethosulfate, N-myristyl-N-methyl morpholinium methyl sulfate, and N-oleyl-N-methyl morpholinium methyl sulfate;
the quaternary morpholinium alkyl sulfate compound being soyethyl morpholinium ethosulfate and/or cetethyl morpholinium ethosulfate;
the quaternary morpholinium alkyl sulfate compound being N-myristyl-N-methyl morpholinium methyl sulfate and/or N-oleyl-N-methyl morpholinium methyl sulfate;
the quaternary morpholinium alkyl sulfate compound being soyethyl morpholinium ethosulfate;
the quaternary morpholinium alkyl sulfate compound being cetethyl morpholinium ethosulfate;
the quaternary morpholinium alkyl sulfate compound being N-myristyl-N-methyl morpholinium methyl sulfate;
the quaternary morpholinium alkyl sulfate compound being N-oleyl-N-methyl morpholinium methyl sulfate;
the air decontamination formulation further comprising a corrosion inhibitor;
the air decontamination formulation further comprising approximately 0.043% to approximately 0.43% by weight of the formulation of a corrosion inhibitor;
the air decontamination formulation further comprising approximately 0.043% to approximately 0.43% by weight of the formulation of a pH adjuster;
the air decontamination formulation optionally further comprising 0% to approximately 0.28% by weight of the formulation of a defoaming agent;
the air decontamination formulation further comprising approximately 0.05% to approximately 0.43% by weight of the formulation of a dye, fragrance, or both;
the air decontamination formulation being substantially free of an emulsifier;
the air decontamination formulation being substantially free of a nonionic surfactant;
the air decontamination formulation being substantially free of an amphoteric surfactant;
the MB nozzle having an actuator orifice with a diameter ranging from approximately 0.016 inches (0.41 mm) to approximately 0.022 inches (0.56 mm);
the MB nozzle having an actuator orifice with a diameter ranging from 0.016 inches (0.41 mm) to 0.022 inches (0.56 mm);
the MB nozzle having an actuator orifice with a diameter ranging from approximately 0.017 inches (0.43 mm) to approximately 0.021 inches (0.53 mm);
the MB nozzle having an actuator orifice with a diameter ranging from 0.017 inches (0.43 mm) to 0.021 inches (0.53 mm);
the MB nozzle having an actuator orifice with a diameter ranging from approximately 0.018 inches (0.46 mm) to approximately 0.020 inches (0.51 mm);
the MB nozzle having an actuator orifice with a diameter ranging from 0.018 inches (0.46 mm) to 0.020 inches (0.51 mm);
the MB nozzle having a stem vapor tap with a diameter ranging from approximately 0.016 inches [0.41 mm] to approximately 0.025 inches [0.64 mm];
the MB nozzle having a stem vapor tap with a diameter ranging from 0.016 inches [0.41 mm] to 0.025 inches [0.64 mm];
the MB nozzle having a stem vapor tap with a diameter ranging from approximately 0.017 inches [0.43 mm] to approximately 0.024 inches [0.61 mm];
the MB nozzle having a stem vapor tap with a diameter ranging from 0.017 inches [0.43 mm] to 0.024 inches [0.61 mm];
the MB nozzle having a stem vapor tap with a diameter ranging from approximately 0.018 inches [0.46 mm] to approximately 0.023 inches [0.58 mm];
the MB nozzle having a stem vapor tap with a diameter ranging from 0.018 inches [0.46 mm] to 0.023 inches [0.58 mm];
the MB nozzle having a stem vapor tap with a diameter ranging from approximately 0.019 inches [0.48 mm] to approximately 0.022 inches [0.56 mm];
the MB nozzle having a stem vapor tap with a diameter ranging from 0.019 inches [0.48 mm] to 0.022 inches [0.56 mm];
the 2-piece MB nozzle producing a spray having a particle size ranging from approximately Dv[50]=45 microns to approximately Dv[50]=90 microns on average as measured using a laser diffractometer;
the 2-piece MB nozzle producing a spray having a particle size ranging from Dv[50]=45 microns to Dv[50]=90 microns on average as measured using a laser diffractometer;
the 2-piece MB nozzle producing a spray having a particle size ranging from approximately Dv[50]=50 microns to approximately Dv[50]=60 microns on average as measured using a laser diffractometer;
the 2-piece MB nozzle producing a spray having a particle size ranging from Dv[50]=50 microns to Dv[50]=60 microns on average as measured using a laser diffractometer;
the 2-piece MB nozzle producing a spray having a particle size ranging from approximately Dv[50]=45 microns to approximately Dv[50]=55 microns on average as measured using a laser diffractometer;
the 2-piece MB nozzle producing a spray having a particle size ranging from Dv[50]=45 microns to Dv[50]=55 microns on average as measured using a laser diffractometer;
the 2-piece MB nozzle producing a spray having a volume moment mean ranging from Dv[4,3]=55 microns to Dv[4,3]=75 microns on average as measured using a laser diffractometer, preferably 60-70;
the 2-piece MB nozzle producing a spray having a volume moment mean ranging from Dv[4,3]=60 microns to Dv[4,3]=70 microns on average as measured using a laser diffractometer.

Methods of sanitizing and disinfecting air in 0.1 to 5 minutes are also disclosed. The MB nozzle of the air decontamination products disclosed above is actuated to introduce between approximately 1.1 g/second to approximately 2.0 g/second of the air decontamination formulations into air for approximately 10 to approximately 60 seconds, the air occupying a volume being less than or equal to 25 m³. The air decontamination formulation may be introduced into the air in one continuous 10 to 60 second spray. Alternatively, the air decontamination formulation may be introduced into the air in multiple consecutive sprays that combine to total 10 to 60 seconds of spray in a total time of less than 15 to 40 seconds. The disclosed methods may include one or more of the following aspects:
introducing the air decontamination formulation into the air for approximately 10 to approximately 45 seconds;
introducing the air decontamination formulation into the air for approximately 10 to approximately 30 seconds.

Methods of sanitizing and disinfecting air in 0.1 to 5 minutes are also disclosed. Between approximately 5 ppm to approximately 30 ppm, preferably between approximately 9 ppm to approximately 12 ppm, of dipropylene glycol vapor is introduced into the air for approximately 10 to approximately 30 seconds, the air occupying a volume being less than or equal to 25 m³. The dipropylene glycol vapor may be introduced in spray form having a particle size ranging from approximately Dv[50]=45 microns to approximately Dv[50]=90 microns, preferably ranging from approximately Dv[50]=50 microns to approximately Dv[50]=60 microns, as measured using laser diffraction.

The above embodiments are exemplary only. Other embodiments as described herein are within the scope of the disclosed subject matter.

### BRIEF DESCRIPTION OF THE SEVERAL VIEWS OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a diagram of the 2-piece MB nozzle aerosol valve assembly that may be used in the present invention;

FIG. 2 is a diagram of liquid electrical vaporizer that may be used in the present invention;

FIG. 3 is a perspective view of an aerobiological testing chamber used in the examples that follow;

FIG. 4 is a flow chart of the steps performed in the following examples;

FIG. 5 is a graph of the log₁₀ concentration of the tested microorganism versus time;

FIG. 6 is a graph of the log₁₀ concentration of the tested microorganism versus time;

FIG. 7 is a graph of the log₁₀ concentration of the tested microorganism versus time;

FIG. 8 is a graph of the log₁₀ concentration of the tested microorganism versus time;

FIG. 9 is a graph of the log₁₀ concentration of the tested microorganism versus time;

FIG. 10 is a diagram of the OVS-7 sorbent tube used to sample the concentration of DGP in the air;

FIG. 11 is an exemplary calibration chromatogram showing the elution time for the three (3) different concentrations of DPG standards, including the peaks for the DPG isomers;

FIG. 12 is a graph of the concentration of DPG in air in ppm versus time in minutes,

FIG. 13 is a graph of the log₁₀ concentration of the tested microorganism versus time, and

FIG. 14 is a graph of the concentration of DPG in air in ppm versus time in minutes.

### DEFINITIONS

As used herein, the term “a” or “an” means one or more.

As used herein, the term “comprising” is inclusive or open-ended and does not exclude any additional elements; the term “consisting of” excludes any additional elements; and the term “consisting essentially of” is in-between, only permitting additional elements that do not materially affect characteristics of the product or process.

As used herein, the term “approximately” or “about” means plus or minus 10 percent of the value stated.

As used herein, the w/w percent of an ingredient is based on the weight of the ingredient in grams in the total weight of the formulation in grams. When an ingredient does not contain close or equal to 100% active material, two percentages may be provided: one for the weight of the ingredient and one for the weight of the active. For example, 0.2 g of Forestall-LQ-(HM) sold by Croda contains approximately 35% soyethyl morpholinium ethosulfate in water, alcohol and/or polypropylene glycol, which equates to approximately 0.07 g of soyethyl morpholinium ethosulfate in the formulation. This would be represented as 0.2 g [0.07 g] of odor neutralizing cationic surfactant.

As used herein, any and all ranges are inclusive of their endpoints regardless of the leading preposition. For example, a pH ranging from 9 to 11 or a pH between 9 and 11 would include formulations having a pH of 9, formulations having a pH of 11, and formulations having any pH between 9 and 11.

As used herein, the phrase “substantially free” means a concentration of less than 0.2 g/L, preferably less than 0.1 g/L, more preferably less than 0.05 g/L, and most preferably less than 0.001 g/L.

As used herein, the terms “germ” and “microbe” or “microbial” means microorganisms which causes disease and encompasses both bacteria and viruses and “microbicidal” means formulations that inactivate (or kill) germs and microbes.

As used herein, the terms “decontamination” or “decontaminate” mean to reduce the concentration of aerosolized microbes in the air. The terms “sanitize”, “disinfect,” “sanitization,” and “disinfection” mean providing equal to or greater than a 3 log₁₀ reduction in 0.1 to 5 minutes of aerosolized microbes, including Staphylococcus aureus and Klebsiella pneumoniae, and aerosolized enveloped viruses, including Phi6, in air occupying a volume less than or equal to 25 m³.

As used herein, the term “vapor” refers to the gaseous state of DPG. The term vapor does not include any droplets suspended in the air but may be formed from such droplets.

As used herein, the term “swirl chamber” may also be known as a spin chamber.

### DETAILED DESCRIPTION

Glycol vapors have been shown to produce decreases in numbers of viable airborne bacteria within enclosed spaces. See, e.g., the US EPA's Efficacy Data and Labeling Requirements: Air Sanitizers, DIS/TSS-11, 3 Sep. 1980; Robertson et al., A Study of the Bactericidal Activity in vitro of Certain Glycols and Closely Related Compounds, The Journal of Infectious Diseases, Vol. 83, No. 2 (September-October 1948), pp. 124-137. The product label for SC Johnson's Oust™ surface disinfectant and air sanitizer lists 6.0% triethylene glycol as one of its active ingredients. As demonstrated in the examples that follow, this triethylene glycol concentration is unlikely to provide air sanitization as defined herein.

Triethylene glycol (TEG) is a colorless, odorless viscous liquid. TEG has a boiling point of 285° C. and density of 1.1255 g/mL. TEG has a vapor pressure of 0.02 Pa at 20° C. TEG is soluble in ethanol and miscible with water. PCT Pub No. WO2007/117534 to SC Johnson (the '534 Publication) discloses that ethanol must be added to TEG-based aerosol compositions to increase its solubility in hydrocarbon propellants. The '534 Publication further discloses that quantities of 12-15% triethylene glycol would produce a two-phase system requiring vigorous shaking by consumers before use.

Dipropylene glycol (DPG) is a colorless, nearly odorless liquid. DPG has a boiling point of 227° C., a density of 1.02 g/mL at 20° C., and a viscosity of 75.0 cPs at 25° C. DPG has a vapor pressure of 2.7 Pa at 20° C. DPG's vapor pressure increases to about 250 Pa just below 100° C. DPG is soluble in ethanol and water. DPG is a mixture of three isomeric chemical compounds: 4-oxa-2,6-heptandiol, 2-(2-hydroxy-propoxy)-propan-1-ol, and 2-(2-hydroxy-1-methyl-ethoxy)-propan-ol. One of ordinary skill in the art will recognize that different isomers may have different properties. Applicant has found that DPG produced by non-catalytic hydration reaction with propylene oxide and water followed by vacuum distillation produces a consistent isomer profile.

Originally, Applicant believed that that air decontamination formulations comprising approximately 28% w/w to approximately 43% w/w dipropylene glycol (DPG), approximately 0.12% w/w to approximately 0.43% w/w odor neutralizing cationic surfactant, approximately 28% w/w to approximately 50% w/w alcohol, and approximately 14% w/w to approximately 33% water were required to provide air disinfection, all weight based on the weight of the air decontamination formulation. The original theory was that “more is better” and higher concentrations of DPG were necessary to achieve germ kill.

Applicant has since discovered that the initial concentration of DPG vapor in the air is more important than the concentration of DPG in the formulation. More particularly, as shown in the examples that follow, germ kill is obtained when the concentration of DPG vapor in the air ranges from approximately 5 to approximately 30 ppm, preferably from approximately 9 to approximately 12 ppm. This concentration is much higher than the theoretical saturation level of 2-3 ppm expected from DPG vapors and substantially higher than the 50% of the theoretical saturation level (i.e., 1-1.5 ppm) recommended by the EPA in DIS/TSS-11 from 3 Sep. 1980. As a result, the DPG vapor concentration in the air decontamination formulation may be as low as approximately 10% by weight, provided that the delivery mechanism generates between approximately 5 to approximately 30 ppm DPG in vapor form. In the examples that follow, this concentration was accomplished by increasing the size of the stem orifice in the aerosol nozzle to produce larger spray particles, ranging from approximately Dv[50]=45 microns to approximately Dv[50] 90 microns, preferably from approximately Dv[50] 45 microns to approximately Dv[50] 55 microns or approximately Dv[50] 50 microns to approximately Dv[50] 60 microns. Alternative delivery mechanisms, such as piezoelectric vaporizers or mist diffusers, may also be used, provided that the approximately 5 to approximately 30 ppm DPG concentration in air is achieved.

The disclosed air decontamination formulations comprise from approximately 10% by weight to approximately 43% by weight dipropylene glycol based on the weight of the formulation, preferably from approximately 11% by weight to approximately 38% by weight, more preferably from approximately 12% by weight to approximately 30% by weight, more preferably from approximately 13% by weight to approximately 25% by weight, and most preferably from approximately 14% by weight to approximately 20% by weight.

The disclosed air decontamination formulations comprise from approximately 0% by weight to approximately 0.2% by weight of an odor neutralizing cationic surfactant based on the weight of the formulation, preferably from approximately 0.05% by weight to approximately 0.15% by weight. Exemplary odor neutralizing cationic surfactants include, but are not limited to, quaternary morpholinium alkyl sulfate compounds, such as soyethyl morpholinium ethosulfate, cetethyl morpholinium ethosulfate, N-myristyl-N-methyl morpholinium methyl sulfate, N-oleyl-N-methyl morpholinium methyl sulfate, or combinations thereof. Soyethyl morpholinium ethosulfate and/or cetyl ethyl morpholinium ethosulfate are particularly preferred odor neutralizing cationic surfactants. Exemplary commercial sources of soyethyl morpholinium ethosulfate include the product sold under the tradename Forestall-LQ-(HM) by Croda. Exemplary commercial sources of cetyl ethyl morpholinium ethosulfate include the product sold under the tradename Barquat cme-A by Lonza.

As stated above, DPG is soluble in both water and ethanol. The percentage of water and alcohol in the disclosed aerosol air decontamination formulations is important to obtain optimal vaporization of both the DPG and odor neutralizing cationic surfactant. The aerosol air decontamination formulations contain (a) a combination of between approximately 33% w/w to approximately 55% w/w alcohol and approximately 38% w/w to approximately 19% w/w water based on the weight of the formulation and (b) a suitable dispensing device. In aerosol dispensing applications using a hydrocarbon propellant, a combination of between approximately 45% w/w to approximately 55% w/w alcohol and approximately 29% w/w to approximately 38% w/w 46.6/33.9 water is particularly preferred, more preferably between approximately 45% w/w to approximately 50% w/w alcohol and approximately 32% w/w to approximately 37% w/w water. Preferably, the formulation contains a weight ratio of alcohol:water of approximately 1:1.3 for aerosol applications. Preferably, the formulation contains a weight ratio of DPG:water:alcohol of approximately 1:2:2.3 for aerosol applications. One of ordinary skill in the art will recognize that the propellant helps to both dry the droplets being dispensed and aerosolize the formulation. Non-aerosol applications will need to be formulated to compensate for DPG's low vapor pressure and removal of the drying effect provided by the aerosol. As a result, a combination of between approximately 7% w/w to approximately 12% w/w alcohol and approximately 0.5% to approximately 4% water may be provided for any dispensing devices that do not utilize a propellant. For non-aerosol applications, the formulation preferably contains a ratio of alcohol:water of approximately 10:1 to approximately 3:1. Alternatively, as shown in the examples that follow, an aqueous solution comprising approximately 10% wt to approximately 43% wt dipropylene glycol generates the approximately 5 ppm to approximately 30 ppm dipropylene glycol in vapor form when using a cool mist ultrasonic humidifier.

Due to its safety profile, ethanol is the preferred alcohol. However, a combination of C₁-C₆ alcohols may be used in the disclosed air decontamination formulations without departing from the teachings herein. For example, the quantity of alcohol in the formulation may comprise 95% ethanol and 5% tert-butyl alcohol or 98% ethanol and 2% isopropanol (e.g., approximately 14% to approximately 47% by weight of the formulation ethanol and approximately 1% to approximately 3% by weight of the formulation t-butyl alcohol). One of ordinary skill in the art will recognize that both alcohols and propellants are volatile organic compounds (VOCs) and therefore the concentrations of both are also selected to remain within any governmental VOC limits.

The disclosed aerosol air decontamination formulations further comprise a pH adjuster to maintain a pH ranging from approximately 9 to approximately 11, preferably from approximately 10 to approximately 10.5. One of ordinary skill in the art will recognize that alkaline formulations are required for compatibility with aerosol cans. Non-aerosol formulations may have neutral pH, ranging from approximately 6 to approximately 8. Exemplary pH adjusters include but are not limited to inorganic bases, alkanolamines, or combinations thereof. Exemplary inorganic bases include hydroxides, such as sodium hydroxide, potassium hydroxide, magnesium hydroxide, ammonium hydroxide, or combination thereof. Exemplary alkanolamines include monoethanolamine, diethanolamine triethanolamine, methylethylhydroxypropylhydroxylamine, or combinations thereof. Buffers may also be used as the pH adjuster, such as glycine and sodium hydroxide, ammonia and ammonium chloride, sodium dihydrogen phosphate and sodium hydroxide, or combinations thereof. The alkanolamines, preferably monoethanolamine, are particularly preferred because they may also help reduce or suppress the rotten egg aroma of hydrogen sulfide, which further enhances the malodour control properties of the disclosed compositions.

Aerosol formulations frequently include a defoaming agent to prevent foaming. Defoaming agents may also be used to solubilize other ingredients, such as the fragrance. The disclosed formulations do not produce a lot of foam and therefore the defoaming agent is not mandatory, unless required for a specific fragrance. The disclosed air decontamination formulations may optionally comprise between 0% to approximately 0.28% by weight defoaming agent. Exemplary defoaming agents include but are not limited to PEG-12 Dimethicone. An exemplary suitable commercial source of PEG-12 dimethicone is sold under the tradename Xiameter™ OFX-0193 Fluid by Dow.

The disclosed air decontamination formulations may optionally further include dye, fragrance, or both.

When provided in a metal or alloy container, the disclosed air decontamination formulations may optionally further comprise a corrosion inhibitor. The corrosion inhibitor decreases the reaction between the air decontamination formulation and the metal or alloy container. The same ingredient may provide both the buffering and corrosion inhibitor capabilities to the formulation. The disclosed aerosol air decontamination formulations may optionally comprise between approximately 0.03% by weight to approximately 0.3% by weight corrosion inhibitor. Exemplary corrosion inhibitors include but are not limited to sodium hydroxide, sodium benzoate, sodium nitrite, sodium silicate, sodium lauryl sarcosinate, borates, or combinations thereof. Exemplary borate corrosion inhibitors may comprise a mixture of monoethanolamine (MEA) borate and monoisopropanol amine (MIPA) borate. An exemplary suitable commercial source of the MEA/MIPA borate corrosion inhibitor is sold under the tradename Crodacor™ BE by Croda.

Alternatively, the metal or alloy container may include an interior coating that prevents reaction between the metal or alloy container and its contents. Exemplary coatings include but are not limited to silicon oxide coatings.

The disclosed formulations are a single liquid phase and therefore do not require shaking by consumers before use. Single phase formulations are also typically easier to manufacture, vaporize and exhibit less stability issues than two phase formulations. There is no criticality to the order of addition of the disclosed formulation ingredients. Specifically, the ingredients that produce the disclosed air decontamination formulations may be mixed in one pot, reducing manufacturing complexity. The resulting formulations have a water-like viscosity (i.e., approximately 0.89 mPa·s at 25° C.).

The compositions disclosed above contain all of the ingredients necessary to obtain germ kill in the air (i.e., DPG, alcohol and water and optionally odour neutralizing cationic surfactant). As a result, the disclosed compositions do not require any added antimicrobial actives. The disclosed compositions are therefore substantially free of cationic biocides, halides, halide-containing compounds, silver, silver-containing compounds, phenols, hydrogen peroxide, sodium hypochlorite, chlorhexidine, quaternary ammonium compounds such as benzalkonium chloride or dialkyl dimethyl ammonium chloride, or any combinations thereof.

The disclosed air decontamination formulations do not require any emulsifiers or amphoteric or nonionic surfactants because the ingredients combine to form a single liquid phase. Emulsifiers may also produce foam, which is undesirable. For example, the disclosed air decontamination formulations are substantially free of partial esters of a polyhydric acid, alcohol or ether, such as glycerine monostearate, sorbitol monostearate, propylene glycol monostearate, or diethylene glycol monostearate. The disclosed air decontamination formulations are substantially free of glyceryl dilaurate, ethylene glycol monopalmitate, propylene glycol monolaurate, ethylene glycol monostearate, or propylene glycol monopalmitate. One of ordinary skill in the art will recognize that fragrances may contain trace amounts of these ingredients. However, any trace amounts included in any fragrances are utilized for the fragrance itself and would not provide sufficient quantities to affect the properties of the disclosed air decontamination formulations. As a result, the disclosed formulations may contain between 0% and approximately 0.1% by weight of a combination of any emulsifiers and amphoteric and nonionic surfactants, more preferably between 0% and approximately 0.075% by weight, and most preferably between 0% and approximately 0.05% by weight.

A particularly preferred air decontamination formulation consists of approximately 10% by weight to approximately 43% by weight of the formulation of dipropylene glycol; approximately 28% by weight to approximately 50% by weight of the formulation of an alcohol, preferably ethanol; approximately 14% by weight to approximately 33% by weight of the formulation of water; optionally 0% by weight to approximately 0.2% by weight of the formulation of an odor neutralizing cationic surfactant; optionally 0% by weight to approximately 0.43% by weight of the formulation of a corrosion inhibitor; optionally 0% by weight to approximately 0.43% by weight of the formulation of a pH adjuster; optionally 0% by weight to approximately 0.29% by weight of the formulation of a defoaming agent; and optionally 0% by weight to approximately 0.43% by weight of the formulation of dye, fragrance, or both.

The disclosed air decontamination formulations may be packaged in a single use or multi-use aerosol canister. Alternatively, the disclosed air decontamination formulation may be packaged in non-aerosol containers and vaporized using mechanical devices, such as piezoelectric vaporizers or mist diffusers. As discussed above, adjustment of the alcohol and water concentrations in the disclosed air decontamination formulations will be necessary for non-aerosol applications due to the low vapor pressure of DPG and drying effect provided by the aerosol.

The disclosed air decontamination formulations reduce the concentration of germs in the air and may sanitize and disinfect air. For testing purposes, the air may be contained in an area less than or equal to 25 m³. One of ordinary skill in the art will recognize that air volume is only provided for comparative testing purposes and that the disclosed formulations may be used in smaller or larger areas without departing from the teachings herein. Between approximately 1.1 g/second to approximately 3.0 g/second, preferably approximately 1.5 g/second to approximately 2.5 g/second, of the aerosolized air decontamination formulations disclosed above is introduced into the air for approximately 10 to approximately 60 seconds, preferably for approximately 10 to approximately 45 seconds, more preferably for approximately 10 to approximately 30 seconds. The air decontamination formulations may be introduced into the air in one (1) spray or multiple sequential shorter sprays.

As shown in the Examples that follow, the disclosed air decontamination formulations provide a 3 log₁₀ reduction of Staphylococcus aureus, Klebsiella pneumoniae, and Phi6 in 0.1 to 5 minutes. S. aureus is one of the more difficult bacteria to eradicate. K. pneumoniae is one of the easier bacteria to eradicate. Phi6 is a bacteriophage that acts as a testing surrogate for enveloped viruses, like SARS-COV-2, corona and influenza viruses. The disclosed air decontamination formulations may also be used to reduce the concentration of small non-enveloped viruses, as demonstrated in the examples that follow with the MS2 bacteriophage. One of ordinary skill in the art will recognize that non-enveloped viruses are more difficult to inactivate than bacteria and enveloped viruses. As a result, the disclosed air decontamination formulations take longer to inactivate MS2 than S. aureus, K. pneumoniae, and Phi6.

The disclosed air decontamination product comprises an aerosol canister comprising the air decontamination formulation and a propellant. The aerosol canister is loaded with the liquid air decontamination formulation and propellant to a pressure approximately equal to or slightly greater than the vapor pressure of the propellant. The air decontamination product comprises approximately 70% by weight to approximately 80% by weight of the air decontamination formulations disclosed above and approximately 20% by weight to approximately 30% by weight of propellant, both based on the total combined weight of the air decontamination formulation and the propellant.

The propellant is a pressurized gas, preferably liquified petroleum gas (LPG). The propellant is not dimethyl ether (DME). DME is incompatible with many packaging components, such as plastic and rubber. LPG comprises a blend of propane and butane sufficient to produce at least 60 psig pressure, preferably from about 70 psig to about 80 psig. For example, the pressurized liquified petroleum gas may comprise approximately 40% to approximately 60% by weight of the propane gas and approximately 40% to approximately 60% by weight of the isobutane gas. One exemplary gas suitable for use with the teachings herein has 42.89% weight propane and 57.11% weight isobutane. This gas is commercially available from multiple vendors as the A-70 hydrocarbon blend having a 70 psig pressure.

As shown in the examples that follow, germ kill is obtained when the concentration of DPG vapor in the air ranges from approximately 5 ppm to approximately 30 ppm, preferably from approximately 9 ppm to approximately 12 ppm. This concentration was accomplished by increasing the size of the stem orifice in the aerosol nozzle to produce larger spray particles, ranging from approximately Dv[50]=45 microns to approximately Dv[50] 90 microns, preferably from approximately Dv[50] 45 microns to approximately Dv[50] 55 microns or approximately Dv[50] 50 microns to approximately Dv[50] 60 microns. Applicant believes that the initial supersaturation of the air with DPG obtained from the disclosed products provides the disclosed germ kill.

The aerosol canister comprises a 2-piece mechanical breakup (MB) nozzle with swirl chamber. The MB nozzle comprises two stem orifices, each of which have a diameter ranging from approximately 0.036 inches [0.91 mm] to approximately 0.044 inches [1.12 mm]. The size of each of the two stem orifices in the disclosed air decontamination product increases the discharge rate of the air decontamination formulation. The size of the stem orifices also increases the particle size of the resulting spray. The 2-piece MB nozzle is commercially available.

By manipulating particle size via the aerosol package configuration, preferential particle sizes may be generated. The ability to generate preferential particle sizes enhances the creation of DPG in the vapor state. The resulting spray has a particle size ranging from approximately Dv[50]=45 microns to approximately Dv[50]=90 microns, preferably from approximately Dv[50]=50 microns to approximately Dv[50]=60 microns or from approximately Dv[50]=45 microns to approximately Dv[50]=55 microns. One of ordinary skill in the art will recognize that Dv[50] is the maximum particle diameter below which 50% of the sample volume exists. There are a wide range of commercially available particle size measuring apparats available, including but not limited to laser diffraction, dynamic light scattering, electrophoretic light scattering, automated imaging, sedimentation, electrozone sensing, or sieving. Laser diffraction, automated imaging, electrozone sensing, and sieving are preferred techniques for the disclosed particle sizes, with laser diffraction particularly preferred.

The resulting spray has a volume moment mean ranging from Dv[4,3]=55 microns to Dv[4,3]=75 microns on average as measured using a laser diffractometer, preferably from Dv[4,3]=60 microns to Dv[4,3]=70 microns. The volume moment mean reflects the size of most of the particles in the sample.

As shown in the examples that follow, testing was performed using two or four stem orifices. While four stem orifices help to increase the flow rate of the disclosed air decontamination formulation, the particle size in the resulting spray was inconsistent and irregular. The size of the two stem orifices in the disclosed air decontamination product produce a less turbulent flow of the needed volume and spray size of air decontamination formulation as compared to four stem orifices.

Applicant was surprised by the required stem orifice and spray particle sizes. One of ordinary skill in the art would not expect that these parameters would successfully produce vaporized glycol. One of ordinary skill in the art would expect that better vaporization would occur using smaller particle spray. The increased discharge rate resulting from the claimed stem orifice size floods the air with vaporized material and particles, producing between approximately 5 ppm to approximately 30 ppm DPG vapors in the air, preferably between approximately 9 to approximately 12 ppm DPG vapors. This air saturation level creates an environment where the glycol partitions onto the germ surface. The DPG creates a liquid film on the surface of any bacterium in the air, resulting in osmotic imbalance of the bacterium and subsequent inactivation. Applicant believes that the DPG prevents the virus particles from replicating.

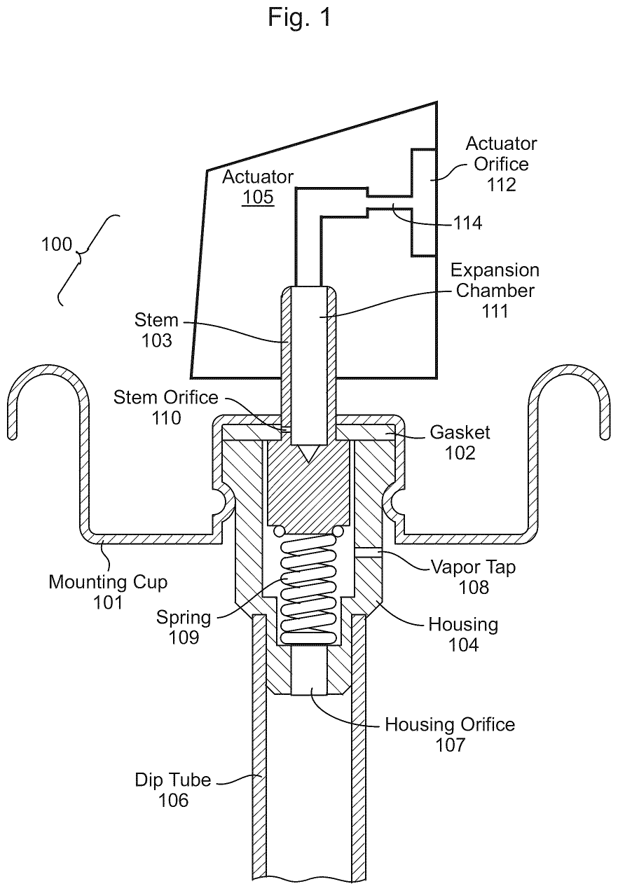

FIG. 1 is a diagram of an exemplary 2-piece MB nozzle aerosol valve assembly 100 that may be used to dispense the disclosed air decontamination formulations. The valve assembly 100 is connected to the mounting cup 101. The mounting cup 101 is crimped to the aerosol canister (not shown) in an air-tight manner. A gasket 102 seals the junction between the top of the valve assembly housing 104, mounting cup 101, and stem 103. The stem 103 extends from the valve assembly housing 104 through the gasket 102 and mounting cup 101 to the actuator 105. The valve assembly housing 104 includes a housing orifice 107, vapor tap 108, and spring 109. The spring 109 holds the valve assembly housing 104 in the closed position. The stem 103 includes a stem orifice 110 and an expansion chamber 111. The single stem orifice 110 in FIG. 1 is provided for illustration purposes only. The two stem orifices 110 in the disclosed product are located on opposite sides of the stem 103. However, the location of the two stem orifices 110 should not have an impact on the resulting particle spray rate and size. The actuator 105 includes a swirl chamber 114 in fluid connection with an actuator orifice 112. The actuator 105 in FIG. 1 depicts a right angle from the stem 103 to the actuator orifice 112. One of ordinary skill in the art will recognize that a straight pathway may also be used without departing from the teachings herein. A dip tube 106 is connected to the bottom of the valve assembly housing 104.

When the actuator 105 is depressed, the stem 103 moves downward, which opens the seal between the gasket 102 and the stem orifice 110. The propellent forces the disclosed air decontamination formulations into the dip tube 106 and through the housing orifice 107 into the valve assembly housing 104. Propellant introduced into the valve assembly housing 104 through the vapor tap 108 mixes with the disclosed air decontamination formulation inside the valve assembly housing 104. The propellant both dries the air decontamination formulation as well as begins formation of aerosol droplets and subsequent glycol vapors. The generation of glycol vapors is absolutely critical in the denaturation of the airborne microbiological species. Applicant has found that the propellant must have at least 60 psig pressure in order to vaporize the disclosed air decontamination formulations. The air decontamination formulation/propellant blend moves from the valve assembly housing 104 through the stem orifice 110 into the expansion chamber 111. From the expansion chamber 111, the formulation moves through the actuator 105 to the swirl chamber 114 and out the actuator orifice 112 as an aerosolized spray.

Numerous swirl chambers 114 are commercially available. See, e.g., U.S. Pat. No. 3,583,642 to SC Johnson & Son, Inc., the contents of which are incorporated herein in its entirety by reference. The swirl chamber 114 is one factor in the production of aerosol particles of the desired size. Initial R&D tests conducted without a swirl chamber 114 resulted in visible spray on the floor below the nozzle. One of ordinary skill in the art will recognize that a pin orifice may also produce a suitably sized aerosol particle.

The size of the actuator orifice 112 combined with the concentration of the propellant in the disclosed air decontamination products provides projection of the spray. The aerosol bloom travels approximately 7 feet (2.1 meters) to approximately 9 feet (2.7 m) from the actuator orifice. This combination permits the larger sized aerosol time to shrink due to evaporation. This combination also permits the aerosol to cover more geography than standard fine mist sprays. The diameter of the actuator orifice 112 ranges from approximately 0.016 inches (0.41 mm) to approximately 0.022 inches (0.56 mm), preferably from approximately 0.017 inches (0.43 mm) to approximately 0.021 inches (0.53 mm), and more preferably from approximately 0.018 inches (0.46 mm) to approximately 0.020 inches (0.51 mm). Alternatively, the diameter of the actuator orifice 112 ranges from 0.016 inches (0.41 mm) to 0.022 inches (0.56 mm), preferably from 0.017 inches (0.43 mm) to 0.021 inches (0.53 mm), and more preferably from 0.018 inches (0.46 mm) to 0.020 inches (0.51 mm).

The size of the vapor tap 108 is another factor that helps determine the size of the aerosol particles. Decreasing the size of the vapor tap 108 lowers the ratio of the propellant to air decontamination formulation and reduces the amount of formulation retention in the canister. But decreasing the size of the vapor tap 108 also increases the aerosol particle size, which may prevent aerosolization of the formulation due to the low vapor pressure of DPG. In other words, as shown in some of the examples that follow, too large an aerosol particle size of the disclosed air decontamination formulations results in liquid being visible on the floor below the canister nozzle. Liquid on the floor does not provide effective air sanitization. As discussed above, increasing the pressure of the propellant permits the larger sized aerosol spray the ability to shrink over distance and time. As a result, the disclosed air decontamination products have a pressure of 60 psig or higher. This difference is most evident in comparison of Examples D and N.

The size of the housing orifice 107 also contributes to the size of the aerosol particles. Decreasing the size of the housing orifice 107 decreases the aerosol particle size.

The size of the stem orifice 110 also contributes to the size of the aerosol particles. Decreasing the size of the stem orifice 110 decreases the aerosol particle size.

The 2-piece MB nozzle aerosol valve assembly 100 comprises an actuator orifice 112 having a diameter ranging from approximately 16 μm to approximately 22 μm, a vapor tap 108 having a diameter ranging from approximately 0.016 inches [0.41 mm] to approximately 0.025 inches [0.64 mm], and two stem orifices 110, each having a diameter ranging from approximately 0.036 inches [0.91 mm] to approximately 0.044 inches [1.12 mm].

Alternatively, the 2-piece MB nozzle aerosol valve assembly 100 comprises an actuator orifice 112 having a diameter ranging from approximately 17 μm to approximately 21 μm, a vapor tap 108 having a diameter ranging from approximately 0.017 inches [0.43 mm] to approximately 0.024 inches [0.61 mm], and two stem orifices 110, each having a diameter ranging from approximately 0.037 inches [0.94 mm] to approximately 0.043 inches [1.09 mm].

In another alternative, the 2-piece MB nozzle aerosol valve assembly 100 comprises an actuator orifice 112 having a diameter ranging from approximately 18 μm to approximately 20 μm, a vapor tap 108 having a diameter ranging from approximately 0.018 inches [0.43 mm] to approximately 0.023 inches [0.61 mm], and two stem orifices 110, each having a diameter ranging from approximately 0.038 inches [0.97 mm] to approximately 0.042 inches [1.07 mm].

In another alternative, the 2-piece MB nozzle aerosol valve assembly 100 comprises an actuator orifice 112 having a diameter ranging from approximately 18 μm to approximately 20 μm, a vapor tap 108 having a diameter ranging from approximately 0.019 inches [0.48 mm] to approximately 0.022 inches [0.56 mm], and two stem orifices 110, each having a diameter ranging from approximately 0.038 inches [0.97 mm] to approximately 0.042 inches [1.07 mm].

As shown in the Examples that follow, the combination of the disclosed air decontamination formulations and the 2-piece MB nozzle aerosol valve assembly 100 sanitizes air in 0.1 to 5 minutes. The actuator 105 is depressed for approximately 10 seconds to approximately 60 seconds to introduce between approximately 1.1 g/second to approximately 2 g/second of the air decontamination formulation into a volume of air occupying less than or equal to 25 m³. The actuator 105 may be depressed manually. Alternatively, the actuator 105 may automatically remain depressed for the desired dispensing duration. For example, the valve assembly 100 may include a solenoid switch as disclosed in PCT Publication Nos. WO2007/045826, WO2007/045827, and WO2007/045828, the contents of which are incorporated herein by reference in their entireties. More particularly, the valve assembly 100 may comprise a moveable magnetic stem 103 surrounded by copper windings (not shown), with an iron frame (not shown) surrounding the copper windings. Electric current applied to the copper windings moves the magnetic stem 103 to either the open or closed position.

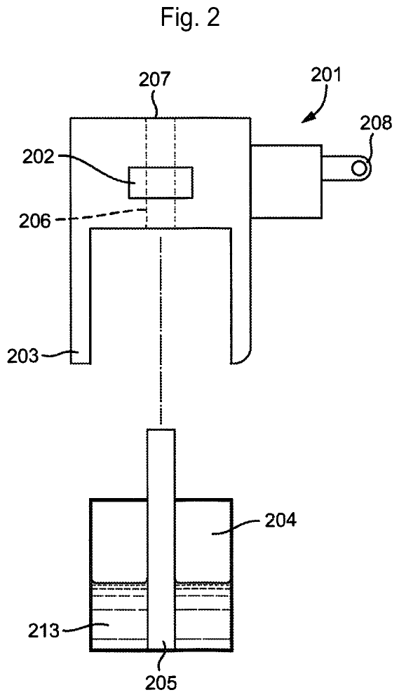

Alternatively, vaporization of the disclosed non-aerosol air decontamination formulations may occur using increased temperature delivered by a liquid electrical vaporizer. The non-aerosol air decontamination formulations comprise or consist essentially of between approximately 84% to approximately 92.5% by weight dipropylene glycol, between approximately 7% to approximately 12% w/w alcohol and approximately 0.5% to approximately 4% by weight water. An exemplary liquid electric vaporizer 201 is shown in FIG. 2, borrowed from FIG. 2 of PCT Pub. No. WO2019/038529 to Reckitt Benckiser (Brands) Limited, the contents of which are incorporated herein in its entirety by reference. The liquid electrical vaporizer 201 typically consists of a housing 203 with electrical plug 208, a heater 202, and a detachable refill bottle 204 with a wick 205 or other liquid transfer mechanism, such as a ceramic coil. The wick 205 may form part of the refill bottle 204. The disclosed air decontamination formulations 213 are contained in the detachable refill bottle 204. The detachable refill bottle 204 typically holds between approximately 20 mL and approximately 25 mL of air decontamination formulation 213, but may easily be adjusted by one of ordinary skill in the art to hold between approximately 100 mL to approximately 500 mL to achieve the purposes disclosed herein. One of ordinary skill in the art will recognize that the single-phase nature of the disclosed air decontamination formulations, and the stability associated therewith, make them particularly suitable for liquid electrical vaporization. Upon insertion of the refill bottle 204 into the housing 203, the wick 205 or ceramic coil is situated close to the heater 202. When the liquid electrical vaporizer 201 is plugged into an electrical outlet, the heater 202 is activated to provide emanation of the air decontamination formulation. The liquid electrical vaporizer 201 of FIG. 2 further comprises a chimney 206. The chimney 206 has an opening 207 through which the vaporized active leaves the vaporizer 201. As discussed above, air sanitization and disinfection occurs when between approximately 1.1 g/second to approximately 2 g/second of the aerosolized air decontamination formulation is introduced into the air for approximately 10 to approximately 60 seconds. Without the aerosol, air sanitization and disinfection is expected to occur when approximately 0.825 g/sec to approximately 1.125 g/sec of the non-aerosolized decontamination formulation is introduced in to the air for approximately 30 to approximately 60 seconds. Non-aerosol applications will need to be formulated to compensate for DPG's low vapor pressure and to remove the requirement for drying provided by the aerosol. As a result, the disclosed formulation may be provided for any dispensing devices that do not utilize a propellant. For non-aerosol applications, the formulation preferably contains a ratio of alcohol:water of approximately 10:1 to approximately 3:1.

One of ordinary skill in the art will recognize that multiple liquid electric vaporizers exist and may be used with the disclosed air decontamination formulations without departing from the teachings herein. For example, the chimney 206 of the liquid vaporizer 201 may include a chimney extension extending further on the distal side than on the proximal side in order to help direct flow of the vaporized material away from the wall and into the room. The liquid vaporizer 201 may also or alternatively include electronic control means to increase the power that is applied to the heater 202 and, consequently, the rate of emanation of the vaporized air decontamination formulation as disclosed in PCT Pub. No. WO2011/045615 to Reckitt & Colman (Overseas) Limited, the contents of which are incorporated herein in its entirety by reference. The electronic control means may further be programmed to provide sufficient heat for approximately 30 to approximately 60 seconds of vaporization at intermittent time intervals, such as every 4, 8, 12, or 24 hours.

The following example below illustrates exemplary embodiments of the invention. It is to be understood that these examples are provided by way of illustration only and that further embodiments may be produced in accordance with the teachings of the present invention.

### EXAMPLES

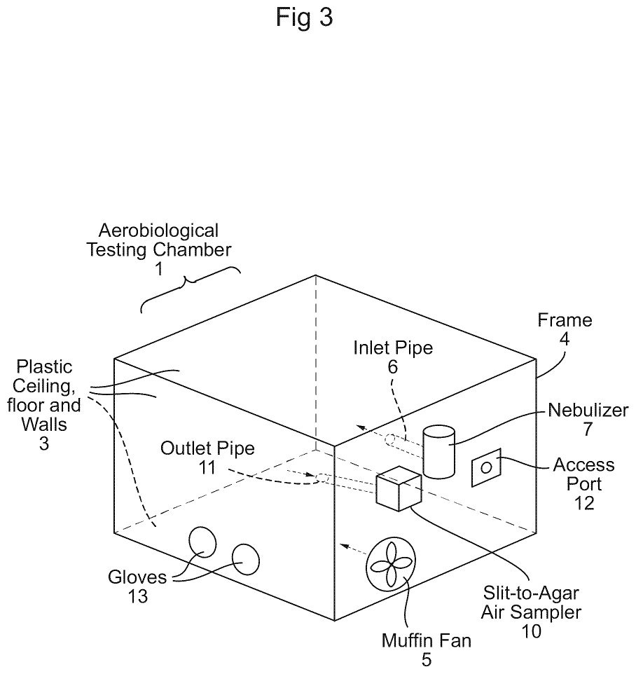

The following examples were performed in the aerobiological testing chamber 1 as shown in FIG. 3 (which is not to scale). The aerobiological testing chamber 1 has a volume of approximately 24 m³ to approximately 25 m³. The chamber 1 is located inside a clean room (not shown) with negative pressure and control access. The ceiling, floor, and walls of the chamber 1 are made of a wipeable white corrugated plastic sheet 3 affixed to the frame 4 of the chamber 1 to maintain an airtight seal preventing air exchange between the chamber 1 and the clean room. While other colors may be used without departing from the teachings herein, the white corrugated plastic sheets provide better visibility for any residual product stains. The wipeable corrugated plastic sheet 3 used in the examples that follow was 0.0157 inches (0.4 cm) thick and sold at Home Depot under the Coroplast™ brand by Coroplast Fritz Müller Gmbh & Co. The walls should be grounded to dissipate any static electricity that may accumulate. A static electricity discharge wire (not shown) is provided to help discharge any static electricity buildup. The chamber 1 may further include clear plastic windows, sealable access doors, and inlets and outlets for the HEPA filtration system. More detailed depictions of some exemplary chambers 1 may be found in Sattar et al., Spread of viral infections by aerosols, Crit Rev Environ Control, 1987, 17:89-131; Sattar et al., Decontamination of indoor air to reduce the risk of airborne infections: Studies on survival and inactivation of airborne pathogens using an aerobiology chamber, American Journal of Infection Control, 44 (2016) e177-e182, the contents of both being incorporated herein by reference in their entireties.

A muffin fan 5, also known as an axial flow fan, is placed on the floor inside the chamber 1 directly underneath the 3.8 cm diameter inlet pipe 6 to a 6-jet nebulizer 7. The 6-jet collision nebulizer 7 generates microbial aerosols in the respirable range of 0.5-5.0 μm. The nebulizer 7 used in the following examples was Model MRE CN24/25 purchased from CH Technologies of Westwood, NJ, US. The nebulizer 7 was connected to a cylinder of extra-dry compressed air with pressure regulator and backflow preventer (neither shown). The fan 5 used was a Nidec Alpha V, TA300, Model A31022-20, Part number 933314 3.0 inch/7.62 m diameter, output 30 CFM supplied by Nidec Corp of Braintree, MA, US. A data recorder (not shown) records the chamber's relative humidity and air temperature. The following examples used the RTR-503L model of wireless data loggers from CAS Data Loggers of Chesterland, OH, US. A magnehelic (not shown) records the pressure differential between the inside and the outside of the chamber 1. The following examples used a magnehelic purchased from ITM Instrument Inc. of Ontario, Canada.

The air in the chamber 1 is sampled at the rate of 1 ft³ (28.3 L)/minute using an externally placed slit-to-agar air sampler with a built-in vacuum pump 10. The sampler 10 used in the examples was purchased from PinPoint Scientific of Bridgend, Wales. The air exiting the sampler 10 is captured in a HEPA filter incorporated in the device 10 or discharged directly into the facility's HEPA-filtered exhaust system (not shown). The sampler 10 draws air samples from the center of the chamber 1 through a 5.0 cm diameter outlet pipe 11.

As discussed by Zargar et al. at page S136, the fan 5 provides uniform distribution of the aerosolized particles in the air inside the chamber 1 when placed at a 45° angle at the bottom of one side of the chamber 1 and operated at 2800 RPM. Mathematical modeling and simulation of bacterial distribution in an aerobiology chamber using computational fluid dynamics, American Journal of Infection Control 44 (2016) S127-S137, incorporated herein in its entirety herein by reference. Zargar et al. further disclose that a 5-minute post-nebulization time is sufficient to distribute introduced bacteria aerosols uniformly throughout the chamber. Id. Zargar et al. further disclose that collection of air samples from the center of the chamber 1 was sufficient to provide a representative profile of the concentration of the airborne bacteria present within the chamber 1. Id.

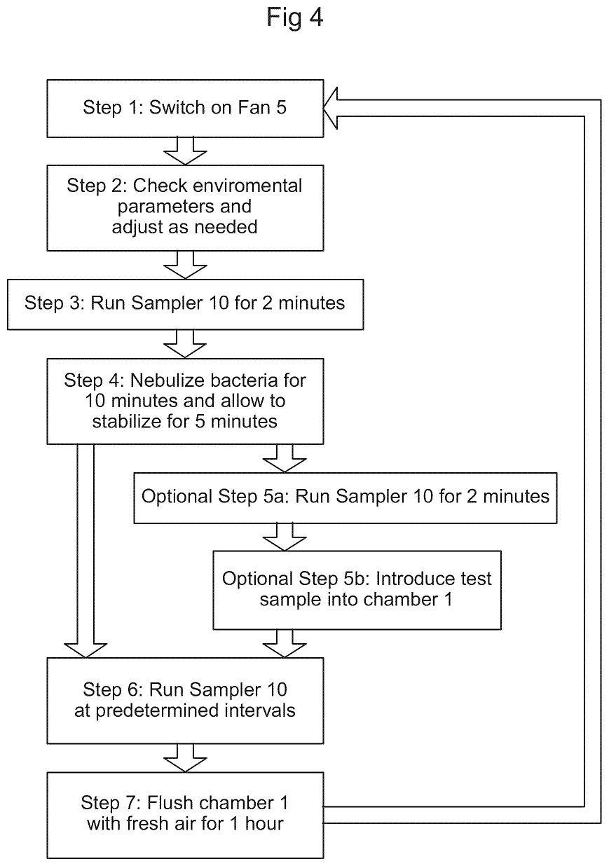

FIG. 4 is a flow chart of the steps performed in the following examples. In step 1, the fan 5 is turned on to start air circulation within the chamber 1. In step 2, the environmental parameters inside the chamber 1 are reviewed and adjusted as needed. Step 3 provides for activation of the slit-to-agar sampler 10 for 2 minutes in order to obtain a sample of any background contamination in the chamber 1. After the sampler 10 is turned off, the bacteria, virus, or phage to be tested is introduced into the chamber 1 via the nebulizer 7 and inlet pipe 6. In this step 4, the nebulizer 7 is run for 10 minutes. The nebulizer 7 is then turned off and the bacteria, virus, or phage allowed to stabilize inside the chamber 1 for 5 minutes. Step 4 should produce a concentration of bacteria, virus, or phage between a minimum of 4.2 log₁₀/m³ to a maximum of 5.0 log₁₀ CFU/m³ in chamber 1.

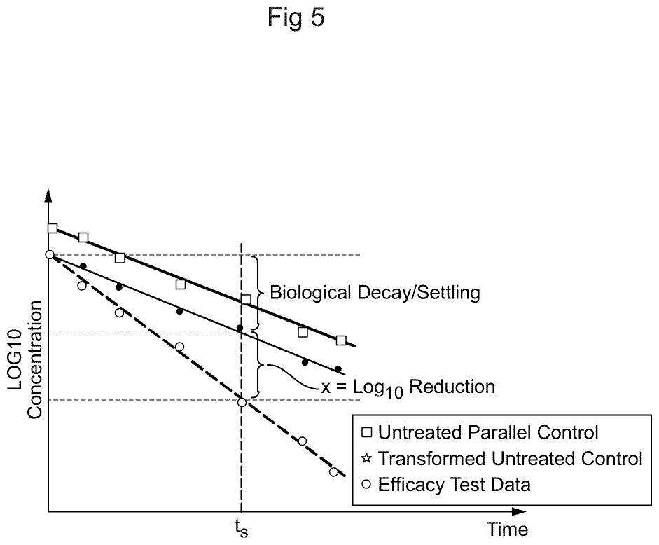

Any meaningful assessment of air decontamination requires that the aerosolized challenge microbes remain viable in the air long enough to allow for proper differentiation between its biological decay or physical fallout and inactivation or removal by the technology being assessed. The test microbes (i.e., bacteria and bacteriophages) were aerosolized into the chamber 1 in Step 4. Steps 5a and 5b are initially skipped to provide the comparative baseline reading of the biological decay or physical fallout. In step 6, samples are collected using a slit-to-agar sampler 10 at predetermined intervals over a specified time frame, for example every 2 minutes over an 8-hour period. The culture plates were incubated at 36° C.±1° C., the colony forming units (CFU) or plaque forming units (PFU) recorded, and the data analyzed to determine the rate of biologic decay. The results are shown in FIG. 5. The chamber 1 was then flushed with fresh air for one hour in Step 7 to decontaminate it and the process started over at Step 1.

Steps 1 to 4 are repeated for different product samples. In Step 5a, the slit-to-agar sampler 10 is run for 2 minutes to determine the initial concentration of the challenge microbes. In Step 5b, the decontamination product to be tested may be introduced into the chamber 1 through an access port 12 in the wall 2 of the chamber 1. Alternatively, the decontamination sample may be placed in the chamber 1 prior to step 1 and accessed and activated using gloves 13 affixed to the wall 2 of the chamber 1 in step 5b. In step 6, samples are collected using a slit-to-agar sampler 10 at predetermined intervals over a specified time frame, for example every 2 minutes over an 8-hour period. The interval and time period for the determination of the biological decay and physical fallout should match the interval and time period used to determine inactivation using the test product. The culture plates were incubated at 36° C.±1° C., the colony forming units (CFU) or plaque forming units (PFU) recorded, and the data analyzed to determine the rate of inactivation. The results are shown in FIG. 5. The chamber 1 was then flushed with fresh air for one hour in Step 7 to decontaminate it and the process started over with a new sample at Step 1.

FIG. 5 is an exemplary graph of the log₁₀ concentration of the tested microorganism versus time. The bold solid lines shows the rate of biological decay of the challenge bacterial species. The dotted line shows the rate of inactivation of the challenge bacterial species by the test product. Since the initial titers of the two experiments may differ, the data from the biological decay is transformed so that its initial titer becomes equal to the initial titer for the inactivation test. This transformation step is easily accomplished by one of ordinary skill in the art. The modified biological decay is shown by the thin solid line. Successful disinfection results are obtained when the log₁₀ reduction from natural or biological decay (thin solid line) to inactivation (dotted line) is equal or greater than 3 log₁₀ after a 5 minute or less contact time tₛ.

The lyophilized test microbes are obtained from a reputable source, such as the American Type Culture Collection (ATCC). The microbes are isolated using standard techniques. The bacteria are cultured to provide approximately 1.6×10⁴ CFU/m³ to approximately 1.0×10⁵ CFU/m³. The nebulization fluid is prepared by adding 50 μL of the cultured bacteria, 0.75 mL Bovine Serum Albumin (BSA), 1.05 mL yeast extract, 3.0 mL mucin, and 10 μL of Antifoam A (from Sigma-Aldrich, Cat A-5633) to 10.14 mL of Dulbecco's Phosphate-Buffered Saline (PBS).

The formulations in the following examples were prepared using the ingredients identified in Table A:

TABLE A

| Abbreviation | CAS | Description |
|---|---|---|
| H₂O | 7732- | Deionized Water |
| | 18-5 | |
| CI | 26038- | Corrosion inhibitor, e.g., MEA Borate and MIPA Borate sold |
| | 87-9 | under the tradename Crodacor ™ BE by Croda |
| | and | |
| | 26038- | |
| | 90-4 | |
| MEA | | Monoethanolamine |
| EtOH | 67-17- | Denatured Ethanol, e.g., SDA 40B sold by MGP |
| | 5 | |
| DPG | | DiPropylene Glycol, e.g., Regular Grade Dipropylene Glycol |
| | | sold by Dow |
| TEG | 112- | TriEthylene Glycol |
| | 27-6 | |
| OFX | | PEG-12 Dimethicone, e.g., Xiameter OFX-0193 Fluid sold by |
| | | Dow |
| SME | | Soyethyl Morpholinium Ethosulfate, e.g., Forestall-LQ-(HM) |
| | | sold by Croda at 35% purity in water, alcohol and/or |
| | | polypropylene glycol |
| Quat | 68989- | Alkyl dimethyl benzyl ammonium saccharinate, e.g., Oxynide |
| | 01-5 | 3300 sold by Stepan |
| F | Various | Fragrance |
| Prop | | Propellant blend of 25.86% w/w propane and 74.14% w/w |
| NP-46 | | N-butane that has a vapor pressure of 46 psig |
| Prop | | Propellant blend of 21.02% w/w propane and 59.32% w/w |
| 152a/AB-46 | | isobutane that has a vapor pressure of 66 psig |
| Prop A-70 | | Propellant blend of 42.89% w/w propane and 57.11% w/w |
| | | isobutane that has a vapor pressure of 70 psig |

The formulations in the following examples were tested using the aerosol nozzles identified in Table B, all of which utilized 2-piece Mechanical Break-up actuators (“2-piece MB”) with swirl chambers.

TABLE B

| | V1 | V2 | V3 | V4 | V5 |
|---|---|---|---|---|---|
| Valve Vapor Tap in | 0.018 | 0.016 | 0.032 | 0.016 | 0.020 |
| inches [mm] | [0.46] | [0.41] | [0.82] | [0.41] | [0.51] |
| Valve Housing Orifice | 0.025 | 0.025 | 0.032 | 0.025 | 0.025 |
| in inches [mm] | [0.64] | [0.64] | [0.81] | [0.64] | [0.64] |
| Valve Stem Orifice, | 4 × 0.024 | 2 × 0.025 | 4 × 0.024 | 2 × 0.025 | 2 × 0.04 |
| number of orifices and | [0.61] | [0.64] | [0.61] | [0.64] | [1.02] |
| diameter of each | | | | | |
| orifice in inches [mm] | | | | | |
| Actuator Exit Orifice in | 0.025 | 0.020 | 0.025 | 0.020 | 0.019 |
| inches [mm] | [0.64] | [0.51] | [0.64] | [0.51] | [0.48] |
| Actuator Lan Length in | 0.031 | 0.010 | 0.025 | 0.040 | 0.020 |
| inches [mm] | [0.79] | [0.25] | [0.64] | [1.02] | [0.51] |

### Example 1: S. aureus

R&D samples of the following formulations were tested against Staphylococcus aureus (ATCC 6538).

The formulations and aerosol nozzles used in the testing are provided in Table 1:

TABLE 1

| | | | EtOH | | | | | | | |
|---|---|---|---|---|---|---|---|---|---|---|
| | | | (190 | | | | SME | | | |
| Ref | H₂O | DPG | Proof) | CI | MEA | OFX | (35%) | F | Prop | Valve |
| A | 39.45 | 25 | 10 | 0.1 | 0.1 | 0.05 | 0.2 | 0.1 | 25 152a/ | V3 |
| | | | | | | | (0.07) | | AB-46 | |
| B | 26.6 | 7.5 | 40 | 0.2 | 0.2 | 0.075 | 0.3 | 0.125 | 25 | V1 |
| | | | | | | | (0.1) | | NP-46 | |
| C | 24.1 | 10 | 40 | 0.2 | 0.2 | 0.075 | 0.3 | 0.125 | 25 | V1 |
| | | | | | | | (0.1) | | NP-46 | |
| D | 19.1 | 15 | 40 | 0.2 | 0.2 | 0.075 | 0.3 | 0.125 | 25 | V1 |
| | | | | | | | (0.1) | | NP-46 | |
| E | 19.45 | 25 | 30 | 0.1 | 0.1 | 0.05 | 0.2 | 0.1 | 25 | V4 |
| | | | | | | | (0.07) | | NP-46 | |
| F | 19.45 | 25 | 30 | 0.1 | 0.1 | 0.05 | 0.2 | 0.1 | 25 152a/ | V1 |
| | | | | | | | (0.07) | | AB-46 | |
| G | 19.45 | 25 | 30 | 0.1 | 0.1 | 0.05 | 0.2 | 0.1 | 25 152a/ | V3 |
| | | | | | | | (0.07) | | AB-46 | |
| H | 19.45 | 25 | 30 | 0.1 | 0.1 | 0.05 | 0.2 | 0.1 | 25 152a/ | V4 |
| | | | | | | | (0.07) | | AB-46 | |
| I | 19.45 | 25 | 30 | 0.1 | 0.1 | 0.05 | 0.2 | 0.1 | 25 | V4 |
| | | | | | | | (0.07) | | A-70 | |
| J | 9.45 | 35 | 30 | 0.1 | 0.1 | 0.05 | 0.2 | 0.1 | 25 | V3 |
| | | | | | | | (0.07) | | A-70 | |
| K | 9.45 | 35 | 30 | 0.1 | 0.1 | 0.05 | 0.2 | 0.1 | 25 | V4 |
| | | | | | | | (0.07) | | A-70 | |
| L | 19.53 | 25 | 30 | 0.07 | 0.1 | 0 | 0.2 | 0.1 | 25 | V2 |
| | | | | | | | (0.07) | | A-70 | |
| M | 19.53 | 25 | 30 | 0.07 | 0.1 | 0 | 0.2 | 0.1 | 25 | V4 |
| | | | | | | | (0.07) | | A-70 | |
| N | 25.46 | 14 | 35 | 0.07 | 0.1 | 0 | 0.2 | 0.17 | 25 | V5 |
| | | | | | | | (0.07) | | A-70 | |

The particle size generated by Formula N was measured using a Malvern laser diffractometer with a 300 mm lens 6 inches from the beam. The average Dv(50) of 3 separate measurements was 54.83 microns. The average volume moment mean D[4,3] of 3 separate measurements was 63.88 microns.

Spray efficacy was demonstrated by obtaining a 3 log₁₀ reduction of Staphylococcus aureus. When available, the spray time, temperature, percent relative humidity (% RH), and results are provided in Table 2:

TABLE 2

| | Spray | Wt. | | | | (a) Log₁₀ | (b) Time | | |
|---|---|---|---|---|---|---|---|---|---|
| | Time | Formulation | Wt. DPG | | | reduction/ | (minutes)/ | | Spray |
| | (In | Sprayed | Sprayed | Temp | | time | 3-LR | Achieve | Visible |
| Ref | Seconds) | (g) | (g) | (° C.) | % RH | (minutes) | achieved? | Sanitization? | on Floor? |
| A | N/A | N/A | N/A | N/A | N/A | N/A | N/A/No | No | N/A |
| B | 60 | 86.78 | 6.5 | 22.5 | 49 | 4.30/120 | 55/Yes | No | No |
| C | N/A | N/A | N/A | N/A | N/A | N/A | N/A/No | No | N/A |
| D | 10 | 11.24 | 1.7 | 23 | 51 | 0.12/105 | 120/No | No | Yes |
| D | 60 | 51.19 | 7.7 | 22.7 | 54 | 0.40/120 | 120/No | No | Yes |
| E | N/A | N/A | N/A | N/A | N/A | N/A | N/A/No | No | N/A |
| F | 15 | 13.53 | 3.4 | 23 | 51 | 3.31/120 | 65/Yes | No | No |
| F | 15 | 11.53 | 2.9 | 23 | 51 | 0.8/120 | 120/No | No | No |
| F | 15 | 12.78 | 3.2 | 22 | 50 | 1.1/120 | 120/No | No | No |
| F | 15 | 9.65 | 2.4 | 23 | 52 | 1.4/120 | 120/No | No | No |
| F | 30 | 17.29 | 4.3 | 22.5 | 47 | 4.21/120 | 3.70/Yes | Yes | No |
| F | 60 | 60.56 | 15.1 | 23 | 45 | 4.34/120 | 4.37/Yes | Yes | No |
| G | N/A | N/A | N/A | N/A | N/A | N/A | N/A/Yes | Yes | N/A |
| H | N/A | N/A | N/A | N/A | N/A | N/A | N/A/Yes | Yes | N/A |
| I | 15 | 12.81 | 3.2 | 22 | 55 | 1.66/120 | 120/No | No | No |
| I | 20 | 18.77 | 4.7 | 23 | 49 | 2.48/120 | 10/Yes | No | No |
| I | 25 | 20.70 | 5.2 | 21 | 53 | 3.97/120 | 5.17/Yes | No | No |
| I | 25 | 19.92 | 5.0 | 25.4 | 53 | 2.86/120 | 5.75/Yes | No | No |
| I | 30 | 32.77 | 8.2 | 22.5 | 53 | 4.11/120 | 3.91/Yes | Yes | No |
| I | 30 | 22.28 | 5.6 | 24.4 | 51.5 | 3.86/120 | 6.32/Yes | No | No |
| I | 30 | 36.68 | 9.2 | 24.4 | 51.5 | 4.06/120 | 3.91/Yes | Yes | No |
| J | 20 | 18.29 | 6.4 | 25.7 | 54 | 1.70/120 | 120/No | No | No |
| K | N/A | N/A | N/A | N/A | N/A | N/A | N/A/No | No | N/A |
| L | 20 | 26.19 | 6.5 | 24.5 | 55 | 1.5/5 | N/A/No | No* | |
| | | | | | | 2.5/105 | | | |
| L | 30 | 37.67 | 9.4 | 24.5 | 54 | 4.50/120 | 4.12/Yes | Yes | No |
| M | N/A | N/A | N/A | N/A | N/A | N/A | N/A/Yes | Yes | N/A |
| N | 30 | 30.21** | 4.2** | 23.9** | 49** | 3/2.86** | 2.86** | Yes | No |

N/A—this data is not currently available
*Small droplets below the can, but not on floor - may be condensation
**average of 3 runs

Table 2 provides both (a) the time it takes to obtain the specified log₁₀ reduction of Staphylococcus aureus and (b) the time it takes to obtain a 3 log₁₀ reduction in Staphylococcus aureus, if indeed a 3 log₁₀ reduction in Staphylococcus aureus was obtained. The formulation is considered a successful air disinfectant when it obtains a 3 log₁₀ reduction in Staphylococcus aureus in 5 minutes or less.

One of ordinary skill in the art will recognize that microbiological test results are not as consistent as chemical test results. Microbiological test results can vary from test to test, even when all other parameters remain the same. Higher variation in microbiological test results is also to be expected in R&D test environments. One of ordinary skill in the art will further recognize that the concentration of bacteria in the air over time may fluctuate. Additionally, the air being sampled in real time may contain residual bacteria when the concentration of the product is sub-optimal/not efficient enough to quickly kill all the bacteria. In other words, multiple factors may have contributed to the results obtained by Formulation I, which was able to achieve a 3 log₁₀ reduction in S. aureus in 5.75 minutes after a 25 second spray, but the reduction decreased to 2.86 log₁₀ after 120 minutes.

As can be seen, the combination of the low concentration of DPG and the low pressure of Formulations B, C, and D did not produce sufficient vapor pressure to achieve sanitization, notwithstanding the higher concentration of ethanol. Formulation E demonstrates that an increase in the concentration of DPG is not sufficient to overcome the limitations of the low-pressure NP-46 propellant. Initially, optimal results were obtained from aerosol formulations containing a ratio of dipropylene glycol:water:alcohol of approximately 1.25:1:1.5.

The approximate 2:1 alcohol:water formulation D produced visible spray on the floor. Applicant believes the propellant may cause the higher concentration of ethanol to evaporate more quickly. No spray was visible on the floor for the aerosol formulations containing a ratio of alcohol:water of approximately ranging from 1.3:1 to 1.5:1.

The 30 second spray of formula F, I, and N and the 60 second spray of formula F successfully achieved air disinfectant germ kill. The 15 second spray results for formula F demonstrate marginal effectiveness that may occasionally generate 3 log₁₀ reduction. In contrast, repeated testing of formula I and N at 30 seconds produces more consistent effectiveness results. Formula N was also tested against K. pneumoniae (ATCC 4352). ≥3 log₁₀ reduction was achieved in 1.17 minutes in the presence of a soil load. This result is expected because inactivation of K. pneumoniae is easier than inactivation of S. aureus.

Table 2 further demonstrates that increasing the spray time (e.g., 60 seconds for Formula B and D) or the percentage of DPG in the formulation (e.g., 35% in Formulations J and K) were not alone sufficient to provide suitable microbiocidal outcome. Formulations J and K were too viscous to move DPG into the vapor phase, even with 70 psig pressure.

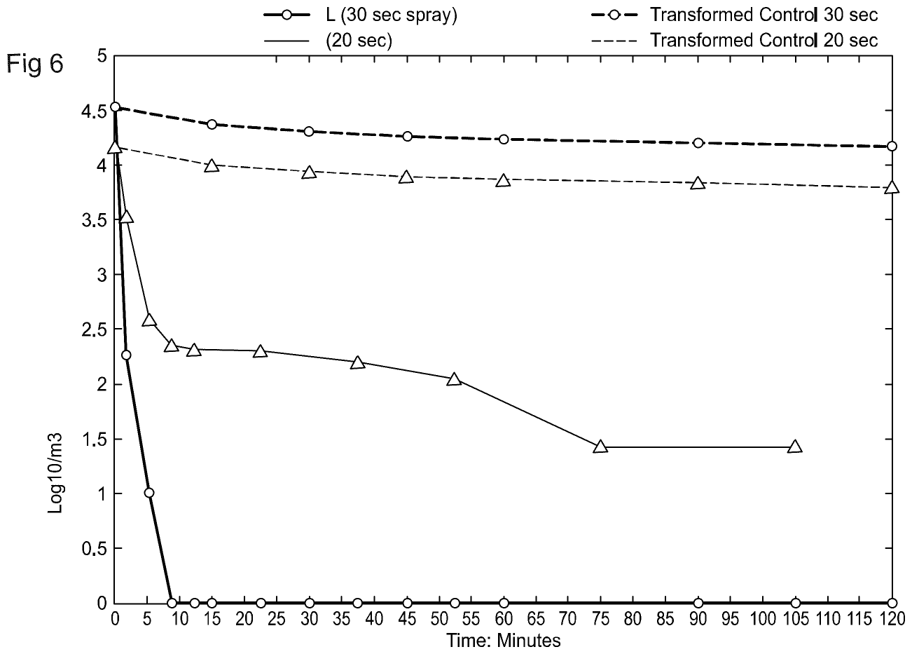

FIG. 6 is a graph of the log₁₀ concentration of S. aureus versus time for Formulation L sprayed for 20 seconds (unbolded line with triangles) and 30 seconds (bolded line with circles). The corresponding dotted lines show the rate of biological decay of the challenge bacterial species. As can be seen, both the 20 and 30 second spray times provide fast reduction in S. aureus concentrations, but only the 30 second spray time provides the 3 log₁₀ reduction in less than 5 minutes.

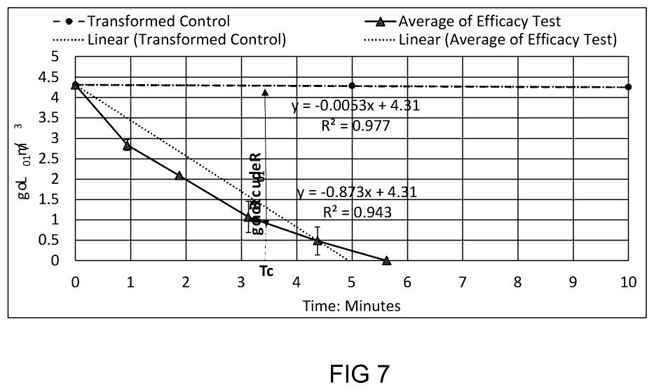

FIG. 7 is a graph of the average log₁₀ concentration of S. aureus versus time for three different lots of Composition N at a 30 second spray time, each lot tested in triplicate (so the average of 9 test results). As can be seen, Composition N obtains a 3 log₁₀ reduction in less than 4 minutes, notwithstanding having a lower concentration of DPG than Composition L. These results demonstrate that concentration of DPG in the air is more important than concentration of DPG in the composition.

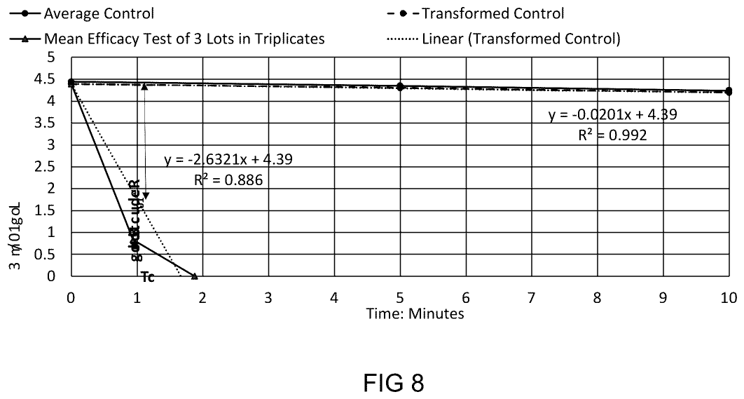

FIG. 8 is a graph of the average log₁₀ concentration of K. pneumoniae versus time for three different lots of Composition N at a 30 second spray time, each lot tested in triplicate (so the average of 9 test results). As can be seen, Composition N obtains a 3 log₁₀ reduction in less than 1 minutes.

One of ordinary skill in the art will recognize that a 20- to 30-second spray time is longer than an average consumer expects to depress a spray button. Applicant expects similar efficacy results after multiple but consecutive shorter spray times. For example, ten (10) consecutive 2- or 3-second sprays or four (4) to six (6) consecutive 5-second sprays, with a <1- to 2-second pause between sprays, are expected to produce similar results.

### Example 2: Phi6 and MS2

Spray efficacy was demonstrated by obtaining a 3 log₁₀ reduction of Phi6 or MS2 after a 30 second spray time. The spray time, temperature, percent relative humidity (% RH), and results are provided in Table 3:

TABLE 3

| | | | | | | Time | |
|---|---|---|---|---|---|---|---|
| | | Wt. | Wt. DPG | | | (minutes) | Spray |
| | | Formulation | Sprayed | Temp | % | to achieve | Visible |
| Ref | Phage | Sprayed (g) | (g) | (° C.) | RH | 3-LR | on Floor? |
| J | Phi6 | 29.69 | 7.4 | 24 | 52 | 4.07 | No |
| J | Phi6 | 32.81 | 8.2 | 23.5 | 49.5 | 3.85 | No |
| J | MS2 | 33.28 | 8.3 | 23.4 | 54 | 14.09 | No |
| J | MS2 | 26.77 | 6.7 | 22.8 | 49 | 13.57 | No |
| N | MS2 | 28.27 | 4.0 | 22.5 | 50 | 9.80 | No |
| N | MS2 | 30.03 | 4.2 | 22.5 | 50 | 11.3 | No |

As discussed above, Table 3 confirms that non-enveloped viruses like MS2 are more difficult to inactivate than bacteria and enveloped viruses like Phi6. That notwithstanding, a 30-second spray of the Formulation J and N is still capable of providing a 3 log₁₀ reduction in concentration.

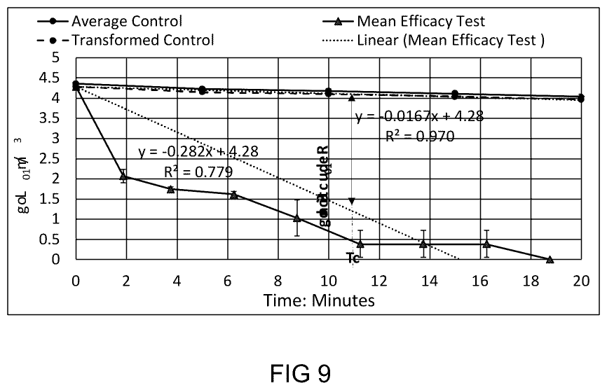

FIG. 9 is a graph of the average log₁₀ concentration of MS2 versus time from Table 3 for the two different lots of Composition N at a 30 second spray time. Each lot was tested in triplicate (so the average of 6 test results). As can be seen, the average of the 6 test results for Composition N obtains a 3 log₁₀ reduction in less than approximately 11 minutes.

### Example 3: DPG vs TEG

Four (4) formulations in Table 4 were tested against S. epidermidis to evaluate the efficacy of DPG versus TEG: TEG Only, DPG Only, TEG-DPG Blend, and TEG-Based. S. epidermidis is a safer, yet equally as relevant as S. aureus, surrogate for a variety of vegetative nosocomial pathogens with potential for airborne spread. Approximately 200 g of product was introduced into the chamber 1 (i.e., the entire canister).

TABLE 4

| | TEG Only | DPG Only | TEG-DPG Blend | TEG-Based |
|---|---|---|---|---|
| H2O | 36.8 | 36.8 | 33.8 | 49.03 |
| DPG | 0 | 3 | 3 | 0 |
| TEG | 3 | 0 | 3 | 5.4 |
| CI | 0.2 | 0.2 | 0.2 | 0.1 |
| EtOH | 40 | 40 | 40 | 20.42 |
| Quat | 0 | 0 | 0 | 0.15 |
| Prop | 20 | 20 | 20 | 24.9 |

The DPG Only and TEG-DPG formulations showed a mean ≥3 log₁₀ reduction from a mean baseline titer of 4.56 log₁₀ after a ≤10 minute exposure. The TEG Only formulation showed a mean log₁₀ reduction of 1.33 at 10 minutes and 2.35 at 60 minutes from a mean baseline titer of 4.83 log₁₀. The TEG-Based formulation showed a mean log₁₀ reduction of 1.59 at 10 minutes and 2.93 at 60 minutes from a mean baseline titer for 4.36 log₁₀. This data demonstrates that DPG is a more effective microbicide in the air than TEG.

### Example 4: DPG Concentration in Air

The concentration of DPG in the air was determined using Capillary Gas Chromatography (GC) with FID detector for Formulation N.

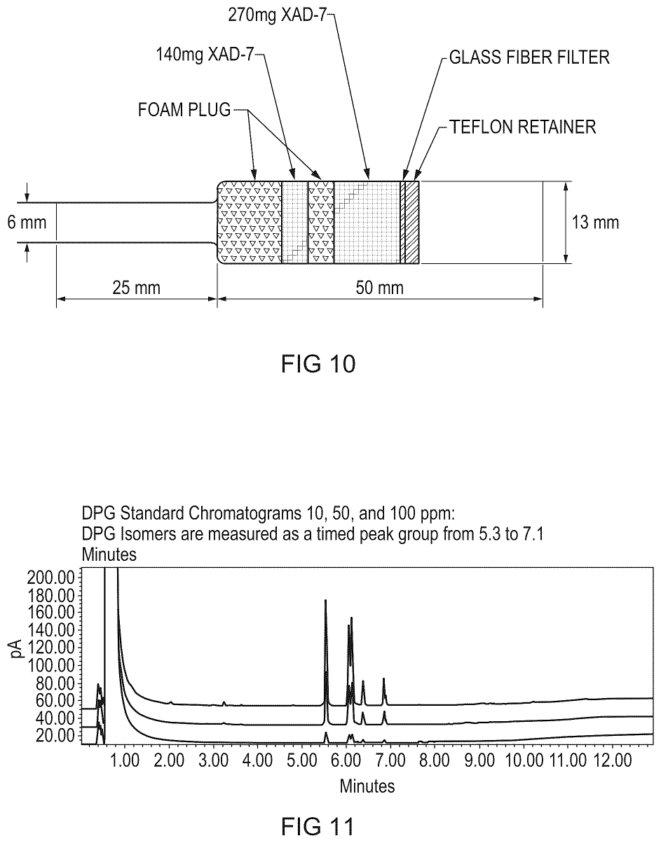

The air samples were collected at the slit-to-agar air sampler 10 of FIG. 3 using a pump, not shown, and custom Occupational Safety and Health Administration (OSHA) Versatile Sampler (OVS) sorbent tubes, particularly configuration 7 (OVS-7), shown in FIG. 10. The pump is calibrated to within +5% of the recommended flow rate.

The custom sorbent tubes had two sections: a 5.0 cm long section with an 11 mm inner diameter and 13 mm outer diameter (o.d.) tapering to a 2.5 cm long section with a 6 mm o.d. A foam plug is inserted into the 13 mm o.d. section until it reaches the junction with the 6 mm o.d. section. 140 mg of hydrophobic organic porous polymer adsorbent, sold under the trade name XAD™-7 by Rohm and Haas, is added to the 13 mm o.d. section to form a layer between the foam plug and a second foam plug is added. An additional 270 mg of polymer adsorbent is added to the 13 mm o.d. section, followed by a glass fiber filler and, finally, a polytetrafluoroethylene (PTFE) retainer. These tubes are currently commercially available as custom products from SKC Inc. (catalog number 226-57), Supelco, and Forest Biomedical.

The 6 mm o.d. end of the custom tube was attached to the pump with flexible tubing. The custom tube was positioned vertically, with the 13 mm o.d. end of the tube pointing obliquely outwards, 76 to 101.6 cm from the floor.

The initial weight of the aerosol can containing formulation N was measured and recorded to two decimals as t⁰. Using the gloves 13, the aerosol was sprayed into the testing chamber 1 for 30 seconds with a sweeping motion towards the center of the chamber. The sampling pump was started and samples taken at the intervals specified in the table below. The room temperature, pressure, relative humidity, final aerosol can weight, and sample volume (liters of air) were recorded for each sample and blank comparison test run.

The test, blank comparison, and standard samples were dissolved in 2 mL isopropanol (IPA). The sample tubes were sealed immediately and allowed to extract/desorb for 30 minutes with either manual shaking or use of a sonicator. A portion of the extracted IPA solution was transferred to 4 ml vials suitable for a GC autosampler. At least 2 injections were run for each sample. The GC was calibrated using a three-point external standard curve, with quantitation ranging from 5 to 300 ppm. FIG. 11 is an exemplary calibration chromatogram showing the elution time for the 3 different concentrations of DPG standards, including the peaks for the DPG isomers.

The DPG concentration (in ppm) of the sample was calculated as (Y-C)/M, where Y is the DPG peak area in the GC chromatogram, C is the y intercept from the GC chromatogram, and M is slope of the line from the 3-point primary standard curve. The total PPM of DPG is the sum of the DPG from the absorbent layers in the front and back of the sample tube. The Replicate Analysis Acceptance Criteria (RAAC) is [(Result of Injection 1/Result of Injection 2)−1]*100, and must be within ±10% to be acceptable. The concentration of DPG in the air sample is calculated as:

$$\frac{( Total ppm DPG ) ( DV ) ( 24.46 ) ( 10 \hat{} 6 ) ( g ) ( mg )}{( Liters ) ( DE ) ( MW ) ( 1000 mg ) ( 1000 µ g )} = ppm \times 5.488 = mg / {M}^{3}$$

where:
   Total ppm DPG=concentration of DPG in sample
   DV=desorption volume=2 mL isopropanol
   24.46=Molar volume (liters/mole) at 25° C. and 760 mm HG
   Liters=liters of air sample
   DE=desorption efficiency=1
   MW=molecular weight (g/mole)=134.17
   5.488=MwDPG/24.45 (unit conversion factor)

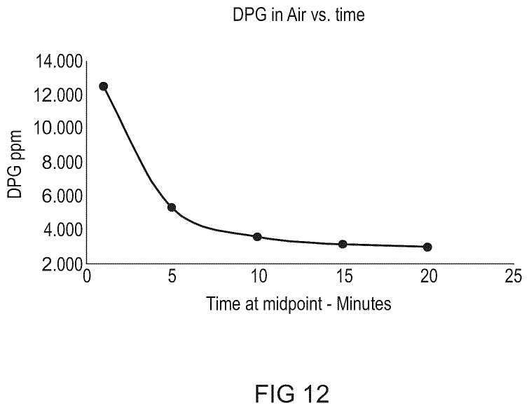

The results are summarized in Table 5 below and FIG. 12.

TABLE 5

| Time Interval | DPG in Sample | | DPG in Air | DPG in Air |
|---|---|---|---|---|
| (in minutes)* | (ppm) | RAAC | (ppm) | (mg/M³) |
| Background | 11.2 | 1.0 | 0.51 | 2.80 |
| 0-2 | 274.1 | −2.4 | 12.49 | 68.56 |
| 4-6 | 116.8 | 2.7 | 5.32 | 29.21 |
| 9-11 | 79.4 | −1.4 | 3.62 | 19.86 |
| 14-16 | 70.1 | 4.2 | 3.20 | 17.53 |
| 19-21 | 66.6 | 8.5 | 3.04 | 16.66 |

*Time is defined as the midpoint of the measurement (e.g., 0-2 minutes = 1, 4-6 minutes = 5, etc)

As can be seen from Table 5 and FIG. 12, the initial concentration of DPG in air measured from 0-2 minutes after spraying the aerosol stopped is approximately 12 ppm. One of ordinary skill in the art will recognize that the concentration of DPG in air will be higher than 12 ppm during the spraying process, particularly towards the end of the spraying process. One of ordinary skill in the art will further recognize the decrease in DPG concentration correlates to the increase in germ kill when FIG. 12 is overlapped with any of FIGS. 6-9. Approximately 10-15 minutes after the spraying is completed, the concentration of DPG in the air achieves an almost steady state of 2-4 ppm, as expected based on the vapor pressure of DPG. This data reinforces that the DPG must be administered in an almost super-saturated vapor state, leading to an instability of the vapor to remain at that concentration. Such scenario leads to rapid partitioning of the vapor onto the external structures of the microbes, helping to create an osmotic shock across the outer bacterial membrane rendering the bacteria non-viable (i.e., killing the bacteria). Lower concentrations of DPG tend to coalesce and settle onto vertical surfaces of treatment areas rather than partitioning onto microbes.

When combined with the data from Table 2, these results confirm Applicant's hypothesis that DPG needs to reach a supersaturated state in order to provide germ kill in the air. These results also confirm that the formulation remains in the air for 2 hours, providing long lasting germ kill and fragrance benefits.

### Example 4: Piezo Deliver

A formulation was prepared containing 18.667% by weight DPG, 34.253% by weight deionized water, and 47.08% by weight ethanol. The mixture produced a single-phase transparent solution. The solution was placed in the Essential Mist Diffuser sold by Reckitt under the Air Wick™ brand, the details of which are disclosed in PCT Pub No WO2017/216548 to Reckitt Benckiser (Brands) Limited. More particularly, the solution was placed in a refill bottle containing a wick formed from polypropylene. The bottle was placed in the atomizer and sprayed successfully, without any spray visible on the atomizer or the surface holding the atomizer. The same formulation was successfully tested in a gravity fed piezo device. Testing to determine the concentration of DPG in the air from these devices is ongoing.

### Example 5: Impact on Risk of Virus Transmission by Aerosols Generated by Toilet Flushing

Pathogen cross-contamination of restrooms by toilet flushing has been widely reported. Applicant tested the effectiveness of Formula N in a real bathroom environment. Bacteriophage MS2 was added to toilet bowl water. As discussed previously, the MS2 bacteriophage is used as a surrogate for non-enveloped viruses, such as human noroviruses. After flushing the toilet, different bathroom surfaces and air were tested for concentrations of MS2. The procedure was repeated with Formula N sprayed for 8 seconds after flushing. A second test was run for no spray and 30 seconds spray of Formula N. The results are provided in Tables 6-8:

TABLE 6

| | No Spray | 8 Seconds Spray of N |
|---|---|---|
| | Conc of MS2 | Conc of MS2 |
| Location | in PFU/cm² | in PFU/cm² |
| Toilet Seat | 1.66 × 10⁸ | 1.12 × 10⁵ |
| Toilet Handle | 1.65 × 10⁴ | 1.29 × 10⁴ |
| Toilet Floor | 1.81 × 10⁶ | 1.4 × 10⁵ |
| Corner Shelf | 2.08 × 10⁵ | 4.67 × 10³ |
| Vanity Top Left Side | 1.45 × 10⁴ | 9.42 × 10³ |
| Vanity Top Right Side | 3.5 × 10⁵ | 2.99 × 10⁵ |

TABLE 7

| | No Spray | 8 Seconds Spray of N |
|---|---|---|
| Location | Conc of MS2 in PFU/m³ | Conc of MS2 in PFU/m³ |
| Air | 6.27 × 10⁵ | 1.9 × 10⁴ |

TABLE 8

| | No Spray | 30 Seconds Spray of N |
|---|---|---|
| | Conc of MS2 in | Conc of MS2 in |
| Location | PFU/cm² | PFU/cm² |
| Toilet Seat | 4.12 × 10⁷ | 2.46 × 10² |
| Toilet Handle | 9.64 × 10³ | 1.87 × 10¹ |
| Toilet Floor | 4.72 × 10⁵ | 1.65 × 10⁴ |
| Corner Shelf | 1.22 × 10⁴ | 1 × 10⁰ |
| Vanity Top Left Side | 5.84 × 10⁴ | 1 × 10⁰ |
| Vanity Top Right Side | 7.93 × 10⁵ | 1 × 10⁰ |

These results demonstrate a significant reduction in cross-contamination of surfaces in potential airborne pathogen exposure from toilet flushing, specifically on the toilet seat after 8 second spray and the corner shelf, left and right vanity top and toilet seat after 30 second spray. An 8 second spray of the disclosed compositions into the air after toilet flushing resulted in an almost 2 log₁₀ reduction in restroom air. Therefore, even using a short 8 second spray of the disclosed compositions after toilet flushing may reduce exposure to common infectious viruses, like norovirus, potentially resulting in reduced risk of infection. This is exciting because norovirus (aka the stomach flu) is very contagious and something no one wants.

### Example 6: Alternative Delivery Mechanism and Bactericidal Activity

The following examples were performed in the aerobiological testing chamber 1 as shown in FIG. 2. The test microorganism, Staphylococcus aureus (ATCC 6538), was prepared using a Trypticase Soy Broth growth medium and TSAM recovery and neutralizing medium (TSA+0.07% lecithin+0.5% polysorbate 80+0.2% sodium thiosulfate). The baseline S. aureus decay was also established as explained in the introductory paragraphs to the Examples and FIG. 3. A cool mist ultrasonic humidifier was used to introduce a deionized water control into the testing chamber 1 at three different durations: 5 minutes, 10 minutes, and 15 minutes. The same model humidifier was used to introduce a 14% wt dipropylene glycol solution in water at four different durations: 5 minutes, 10 minutes, 15 minutes, and 30 minutes. The same model of two different humidifiers was used to reduce need for cleaning between experiments (i.e., one for water alone and a different one for the 14% DPG solution). The results are provided in Table 9 below, with H2O referring to the deionized water control test results and DPG referring to the 14% DPG test results.

TABLE 9

| | | | | | | | 3 Log₁₀ achieved |
|---|---|---|---|---|---|---|---|
| | | | | | | | (Yes/No) |
| | | Weight | | | | | If Yes, Time to |
| | Intro Time | Intro | Intro Rate | Temp | RH | Baseline | Achieve |
| Sample | (minutes) | (g) | (g/minute) | (° C.) | (%) | Log₁₀ | (minutes) |
| H2O | 5 | 20.8 | 4.16 | 23.0 | 46-49 | 4.39 | No |
| DPG | 5 | 20.03 | 4.01 | 23.2 | 48-49 | 4.42 | No |
| H2O | 10 | 33.91 | 3.39 | 23.6 | 49-54 | 4.51 | No |
| DPG | 10 | 24.2 | 2.42 | 23.0 | 46-48 | 4.51 | Yes |
| | | | | | | | 9 minutes |
| H2O | 15 | 56.24 | 3.75 | 21.6 | 50-60 | 4.48 | No |
| DPG | 15 | 62.55 | 4.17 | 21.9 | 49-52 | 4.46 | Yes |
| | | | | | | | 9 minutes |
| DPG | 15 | 62.94 | 4.20 | 19.3 | 51-52 | 4.29 | Yes |
| | | | | | | | 9 minutes |
| DPG | 30 | 125.96 | 4.20 | 20.0 | 52-55 | 4.29 | Yes |
| | | | | | | | 17 minutes |

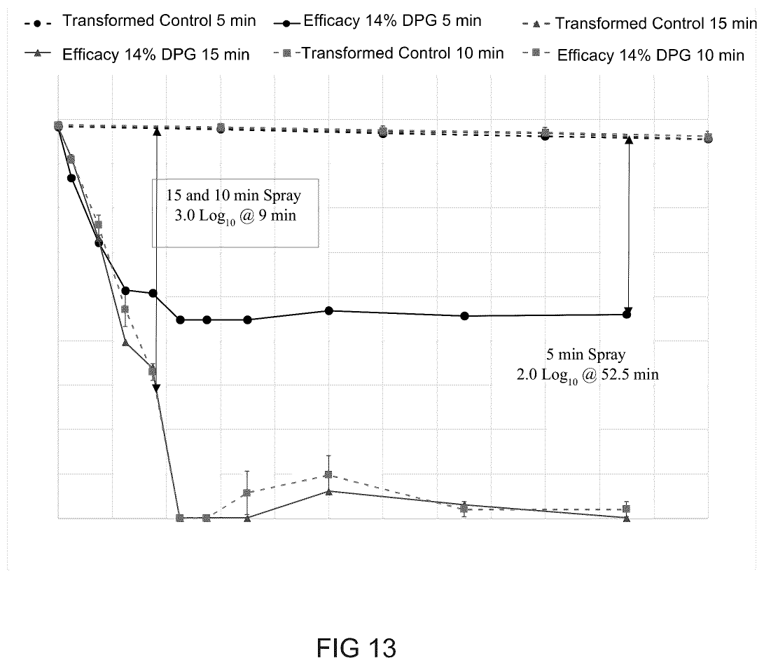

A graph of the log₁₀ concentration of the tested microorganism versus time for some of the DPG results of Table 9 is provided in FIG. 13. The dotted lines provide the transformed control. The solid lines provide the DPG results, with the circle representing the 5-minute DPG introduction, the square representing the 10-minute DPG introduction, and the triangle representing the 15-minute DPG introduction. Both the 10-minute and 15-minute DPG introduction provided 3 log₁₀ reduction of S.aureus in air in approximately 9 minutes. At approximately the same time, the 5-minute DPG introduction provided a 2 log₁₀ reduction. These results demonstrate that the 5-minute DPG introduction did not provide a sufficient quantity of DPG to yield a 3 log₁₀ reduction of S. aureus, but still provided a level of decontamination.

The air sanitization time doubled from 5 minutes for aerosol delivery to approximately 10 minutes using this delivery mechanism. However, the humidifier only generated on average approximately 0.06 g/sec of DPG. Applicant believes that a 3 log₁₀ reduction using humidifier delivery may occur in 5 minutes or less when the delivery system is optimized to provide higher concentrations of DPG in the air. For example, an aqueous solution comprising approximately 25% wt to approximately 43% wt dipropylene glycol may provide such results. A fan may also be added to the humidifier to help better disperse the formulation in the air.

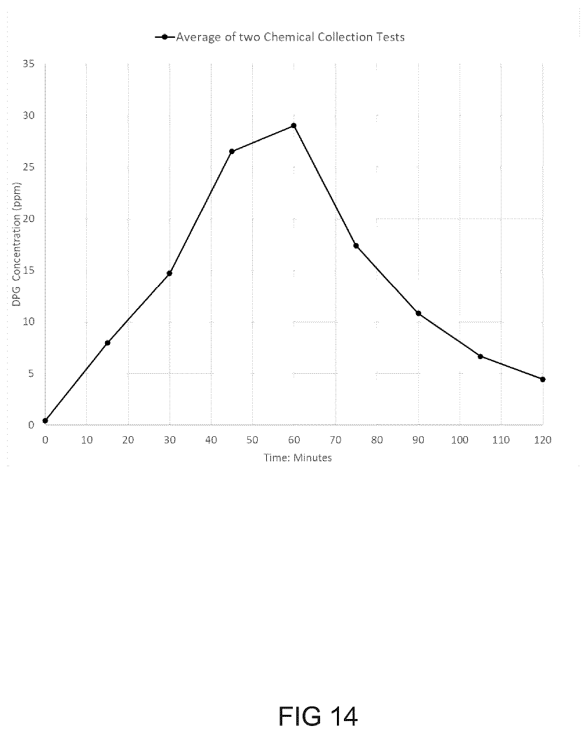

The concentration of DPG in the air of the chamber was measured without the introduction of any S. aureus. The humidifier was turned on for 60 minutes with sampling for 120 minutes, collected at Time 0, 15, 30, 45, 60, 75, 90, 105, and 120 minutes. The results are provided in FIG. 14. As can be seen, the concentration of DPG increased over 5 ppm at just under 10 minutes.

These results further confirm Applicant's hypothesis that between approximately 5 ppm to approximately 30 ppm dipropylene glycol in vapor form provides germ kill in the air.

The foregoing description of various aspects of the invention has been presented for purposes of illustration and description. Embodiments and/or features therein may be freely combined with one another. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A method of sanitizing air in 0.1 to 5 minutes, the method comprising generating between approximately 5 ppm to approximately 30 ppm dipropylene glycol in vapor form.

2. The method of claim 1, wherein an aqueous solution comprising approximately 10% wt to approximately 43% wt dipropylene glycol generates the approximately 5 ppm to approximately 30 ppm dipropylene glycol in vapor form.

3. The method of claim 1, wherein the approximately 5 ppm to approximately 30 ppm dipropylene glycol in vapor form is generated by actuating an aerosol canister comprising
(a) a 2-piece mechanical breakup (MB) nozzle with swirl chamber or break up bar,
(b) an air decontamination formulation comprising:
(i) approximately 10% wt to approximately 43% wt by weight of the formulation of dipropylene glycol;
(ii) alcohol; and
(iii) water, and
(c) pressurized liquified petroleum gas, the pressurized liquified petroleum gas comprising a blend of propane and butane sufficient to produce at least 60 psig pressure;
the MB nozzle comprising two stem orifices, each having a diameter ranging from approximately 0.036 inches [0.91 mm] to approximately 0.044 inches [1.12 mm].

4. The method of claim 3, wherein the air decontamination formulation comprises from approximately 11% wt to approximately 38% wt by weight of the air decontamination formulation.

5. The method of claim 3, wherein the method achieves a 3 log₁₀ reduction of an enveloped virus in 5 minutes or less.

6. The method of claim 3, wherein the method achieves a 3 log₁₀ reduction of a non-enveloped virus in 15 minutes or less.

7. An air decontamination product comprising an aerosol canister comprising
(a) a 2-piece mechanical breakup (MB) nozzle with swirl chamber or break up bar,
(b) an air decontamination formulation comprising:
(i) approximately 10% wt to approximately 43% wt dipropylene glycol by weight of the formulation;
(ii) alcohol; and
(iii) water, and
(c) pressurized liquified petroleum gas, the pressurized liquified petroleum gas comprising a blend of propane and butane sufficient to produce at least 60 psig pressure;
the MB nozzle comprising two stem orifices, each having a diameter ranging from approximately 0.036 inches [0.91 mm] to approximately 0.044 inches [1.12 mm].

8. The air decontamination product of claim 7, wherein the air decontamination formulation comprises from approximately 11% wt to approximately 38% wt dipropylene glycol by weight of the air decontamination formulation.

9. The air decontamination product of claim 7, wherein the air decontamination formulation is a single-phase liquid.

10. The air decontamination product of claim 7, wherein the air decontamination formulation further comprises an odor neutralizing cationic surfactant.

11. The air decontamination formulation of claim 10, wherein the odor neutralizing cationic surfactant is a quaternary morpholinium alkyl sulfate compound.

12. The air decontamination product of claim 7, wherein the air decontamination formulation comprises:
(iii) approximately 35% to approximately 55% by weight of the formulation of ethanol; and
(iv) approximately 29% to approximately 38% by weight of the formulation of water.

13. The air decontamination formulation of claim 7, wherein the air decontamination product comprises approximately 70% by weight to approximately 80% by weight of (b) the air decontamination formulation and approximately 20% by weight to approximately 30% by weight of (c) the pressurized liquid petroleum gas, both based upon the combined weight of (b) the air decontamination formulation and (c) the pressurized liquid petroleum gas.

14. The air decontamination product of claim 7, wherein the MB nozzle further comprises an actuator orifice having a diameter ranging from approximately 0.016 inches [0.41 mm] to approximately 0.022 inches [0.56 mm]

15. The air decontamination product of claim 14, wherein the diameter of the actuator orifice ranges from approximately 0.017 inches [0.43 mm] to approximately 0.021 inches [0.53 mm].

16. The air decontamination product of claim 7, wherein the MB nozzle further comprises a stem vapor tap having a diameter ranging from approximately 0.016 inches [0.41 mm] to approximately 0.020 inches [0.51 mm].

17. The air decontamination product of claim 7, wherein the MB nozzle generates a spray having a particle size ranging from approximately Dv[50]=45 to approximately Dv[50]=90 microns.

18. The air decontamination product of claim 17, wherein the particle size ranges from approximately Dv[50]=50 to approximately Dv[50]=60 microns or from approximately Dv[50]=45 to approximately Dv[50]=55 microns.

19. The air decontamination product of claim 7, wherein the MB nozzle generates a spray having a volume moment mean ranging from approximately Dv[4,3]=55 to approximately Dv[4,3]=75 microns.

20. The air decontamination product of claim 19, wherein the volume moment mena ranges from approximately Dv[4,3]=60 to approximately Dv[4,3]=70 microns.
